(19) United States
(12) Patent Application Publication
(10) Pub. No.: US 2021/0267944 A1
(43) Pub. Date: Sep. 2, 2021

(54) METHODS FOR TREATING AGITATION USING DEXMEDETOMIDINE HYDROCHLORIDE

(71) Applicant: BioXcel Therapeutics, Inc., New Haven, CT (US)

(72) Inventors: Frank D. YOCCA, New Haven, CT (US); Michael DEVIVO, New Haven, CT (US)

(21) Appl. No.: 17/254,982

(22) PCT Filed: Jun. 26, 2019
(86) PCT No.: PCT/US2019/039308

§ 371 Date: Dec. 22, 2020

Related U.S. Application Data
(60) Provisional application No. 62/690,407, filed on Jun. 27, 2018.
(60) Provisional application No. 62/767,422, filed on Nov. 14, 2018.
(60) Provisional application No. 62/787,649, filed on Jan. 2, 2019.
(60) Provisional application No. 62/798,842, filed on Jan. 30, 2019.

(51) Int. Cl. A61K 31/4174; A61K 9/00; A61P 25/20

(52) U.S. Cl. CPC A61K 31/4174; A61P 25/20; A61K 9/0019

(57) ABSTRACT

The present disclosure relates to the treatment of agitation or signs of agitation in certain human subjects, including subjects with a neurodegenerative, neuropsychiatric or opioid withdrawal disorder, by administering dexmedetomidine hydrochloride by the intravenous route.

### CROSS-REFERENCE TO RELATED APPLICATIONS

This application claims priority to U.S. Provisional Application No. 62/690,407, filed Jun. 27, 2018; U.S. Provisional Application No. 62/767,422, filed Nov. 14, 2018; U.S. Provisional Application No. 62/787,649, filed Jan. 2, 2019; and U.S. Provisional Application No. 62/798,842, filed Jan. 30, 2019; each of which is herein incorporated by reference in its entirety for all purposes.

### FIELD OF THE INVENTION

The present disclosure relates to the treatment of agitation or signs of agitation in certain human subjects, including subjects with a neurodegenerative, neuropsychiatric or opioid withdrawal disorder, by administering dexmedetomidine hydrochloride by the intravenous route. The desired anti-agitation effect may be achieved without also producing clinically meaningful effects on blood pressure and/or heart rate.

### BACKGROUND OF THE INVENTION

Agitation is an umbrella term that can refer to a range of behaviors disorders or disorders, including aggression, combativeness, hyperactivity, and disinhibition. Agitation is a nonspecific constellation of relatively unrelated behaviors that can be seen in a number of different clinical conditions, usually presenting a fluctuating course. Agitation may be caused by a number of different medical conditions and drug interactions or by any circumstances that worsen the person's ability to think.

Agitation in elderly patients with dementia, such as senile dementia associated with Alzheimer's Disease (SDAT) and vascular dementia, contributes to additional stress for caregivers and often requires additional treatment with medication. Specific subtypes of agitation include that associated with delirium, psychosis, depression (with or without psychosis), anxiety, insomnia, sundowning (progression of agitation in the evening hours), aggression and anger, and pain (e.g., osteoarthritic). Agitation can also be caused by medication or other substances used to treat the underlying syndrome associated with agitation.

Zeller et. al., discloses in West Journal of Emergency Medicine (2016) 17(2): 165-172 that agitation is frequently associated with psychiatric disorders such as bipolar disorder and schizophrenia. In bipolar disorder, agitation usually present during acute manic states, but can also present during mixed depressive states.

Guthrie S K et. al., disclosed in DICP (1990) July-August; 24(7-8): 721-734, that agitation, especially manifesting as psychomotor agitation, is a symptom of drug (i.e. narcotics) withdrawal. The initial withdrawal symptoms, along with hyper-arousal, anxiety, dysphoria, insomnia, and temperature instability, are caused by central adrenergic system activity. Mello et. al., Journal of Pharmacology, 1982, pages 30-39 mentions that treatment with longer acting opioids, such as buprenorphine and methadone, attenuate the withdrawal syndromes at the opioid receptor, but suffer from potential abuse liability because of their mixed opioid agonist-antagonist properties.

Common medications used to treat agitation include beta blockers such as propranolol and pindolol, anxiety medications such as buspirone, anti-convulsants such as valproate and lamotrigine, anti-psychotics such as haloperidol and other high-potency dopamine-blocking agents and atypical antipsychotics. These medications have limitations because of their delayed action, and prolong administration leads to drug developed tolerance and physical dependence. Antipsychotic drugs such as haloperidol are also associated with extrapyramidal side effects.

The treatment of agitation, and particularly acute agitation, without the various safety concerns of existing options is large unmet medical need. New modalities that provide an appropriate therapeutic outcome in an agitated patient, while avoiding the safety issues associated with existing treatments and the consequent increased burden on the patient and medical community, would represent a significant step forward in anti-agitation therapy.

Dexmedetomidine hydrochloride is a highly selective alpha-2 adrenergic agonist with significant sedative, analgesic, and anxiolytic effects. Dexmedetomidine hydrochloride has been approved by the FDA as an IV infusion product (Precedex®) for sedation of initially intubated and mechanically ventilated adult patients in an intensive care setting and is also approved for sedation of non-intubated adult patients as a component of monitored anesthesia care during surgical or diagnostic procedures. IV dexmedetomidine hydrochloride has also been shown to decrease post-operative emergence agitation in children and elderly populations and achieve a smooth emergence from anesthesia following surgery, especially when sevoflurane is used as the anesthetic.

### SUMMARY OF THE INVENTION

The present disclosure describes the administration of dexmedetomidine hydrochloride by the intravenous route to treat agitation or signs of agitation in a new group of subjects, namely subjects who are susceptible to episodes of agitation, but are alert. Alert subjects are defined herein as individuals who have not recently been administered an anesthetic, such as following surgery, and may still be susceptible to the anesthetic side effects, including emergence agitation. Alert subjects may, for example, experience agitation associated with a neurodegenerative neuropsychiatric, bipolar or opioid withdrawal disorder. Surprisingly, the authors of this disclosure have found particular dosage regimens of IV dexmedetomidine hydrochloride that can be used to treat mild, moderate and severe forms of agitation or signs of agitation in alert subjects without also producing clinically meaningful effects on cardiac function, such as on heart rate and/or blood pressure. The dexmedetomidine hydrochloride may conveniently be administered intravenously as one or more injections or as a continuous infusion to provide the appropriate amount of drug to reduce agitation or signs of agitation in alert subjects. The reduction in agitation may also be associated with a mild sedative effect, such as an effect corresponding to a RASS score of −1.

Thus, the present disclosure relates to methods and compositions for the administration of intravenous dexmedetomidine hydrochloride to treat agitation, preferably acute agitation, or signs of agitation in alert subjects. The disclosure also provides particularly advantageous dosages and dosage regimens of intravenous dexmedetomidine hydrochloride to treat agitation, preferably acute agitation, or signs of agitation in alert subjects. The dosages and dosage regiments may, for example, be effective to treat agitation, preferably acute agitation, or signs of agitation in alert subjects without also producing clinically meaningful effects on cardiac function, such as on heart rate and/or blood pressure. The methods and compositions herein may also advantageously put the patient into a mildly sedated situation that is especially useful in a clinical setting. The methods and compositions may be employed, in particular, on alert subjects suffering from acute agitation or hyper-arousal linked to a medical condition.

In one aspect, the present disclosure provides a method for treating agitation or signs of agitation in a human subject who is alert prior to treatment, comprising administering dexmedetomidine hydrochloride intravenously to the subject at a cumulative dose of about 0.1 mcg/kg to about 1 mcg/kg to achieve the desired anti-agitation effect.

In another aspect, the present disclosure provides a method for treating agitation or signs of agitation in a human subject who is alert prior to treatment, comprising administering dexmedetomidine hydrochloride intravenously to the subject to a total dose in a range from about 2 mcg to about 100 mcg to achieve the desired anti-agitation effect.

The methods and compositions disclosed herein also comprise administering intravenous dexmedetomidine hydrochloride to treat hyper-arousal in alert subjects.

In some aspects, the subject may not have an underlying disease or disorder associated with the agitation (e.g. acute agitation) or hyper-arousal. In other aspects, the agitation or hyper-arousal may be caused by a disease or disorder. For example, the subject with agitation (e.g. acute agitation) may have a neurodegenerative disorder such as senile dementia of the Alzheimer's type (SDAT), a neuropsychiatric disorder such as schizophrenia, bipolar disorder or hyperactive delirium. In other aspects, the agitated or hyper-aroused subject may be suffering from opioid withdrawal (e.g. precipitated withdrawal) or alcohol withdrawal symptoms.

In further aspects, the methods and compositions disclosed herein comprise administering intravenous dexmedetomidine hydrochloride to treat agitation or signs of agitation associated with senile dementia of the Alzheimer's type (SDAT), schizophrenia, opioid withdrawal symptom, bipolar disorder, or hyperactive delirium.

In one aspect, intravenous dexmedetomidine hydrochloride may be administered to alert subjects treated for agitation or signs of agitation associated with SDAT, to achieve a positive response to SDAT symptoms, for example, as measured according to the Richmond Agitation Scale Score (RASS) scale. A positive response equates to a reduction in the RASS score to −1 (“arousable sedation” also referred to herein as a “mild sedative effect” or “mild sedation”).

In another aspect, intravenous dexmedetomidine hydrochloride may be administered to alert subjects treated for agitation or signs of agitation associated with schizophrenia, to achieve a positive response to schizophrenia symptoms, for example, as measured according to the RASS and/or PEC scales. A positive response equates to a reduction in the RASS score to −1 or a PEC score of 7 or below.

In another aspect, intravenous dexmedetomidine hydrochloride may be administered to alert subjects treated for agitation or signs of agitation associated with opioid abuse, to achieve a positive response to opioid withdrawal symptoms, for example, as measured according to the Clinical Opioid Withdrawal Scale (COWS), an 11-item scale that measures a constellation of withdrawal symptoms experienced after abstaining from opioid use. The clinician assigns a numerical score for each characteristic signs and symptoms of opiate withdrawal. The sum total score is used to grade and track the severity of physiological withdrawal from opioids.

In a particular aspect, the methods and compositions disclosed herein may achieve at least a 50% reduction in COWS score.

In some aspects, the alert subject is at least 55, at least 60, at least 65 or at least 75 years of age.

### BRIEF DESCRIPTION OF THE DRAWINGS

FIG. 1: depicts the percentage of SDAT patients achieving RASS of −1.

FIG. 2. depicts the mean Systolic blood pressure vs actual time in Alzheimer's patients infused with dexmedetomidine hydrochloride (treatment arm) and normal saline (placebo arm)—. Semi-log scale.

FIG. 3. depicts the mean Diastolic blood pressure vs actual time in Alzheimer's patients infused with dexmedetomidine hydrochloride (treatment arm) and normal saline (placebo arm)—. Semi-log scale.

FIG. 4. depicts the mean Pulse rate vs actual time in Alzheimer's patients infused with dexmedetomidine hydrochloride (treatment arm) and normal saline (placebo arm)—. Semi-log scale.

FIG. 5. depicts the mean Respiration rate vs actual time in Alzheimer's patients infused with dexmedetomidine hydrochloride (treatment arm) and normal saline (placebo arm)—. Semi-log scale.

FIG. 6. depicts the mean Plasma concentration (pg/ml) vs actual time in Alzheimer's patients infused with dexmedetomidine hydrochloride—Semi-log scale.

FIG. 7: depicts the percentage of schizophrenic patients achieving RASS −1 in the treatment arm (dexmedetomidine treated group) versus placebo group.

FIG. 8: depicts the mean drop in PEC score in schizophrenic patients in the treatment arm (dexmedetomidine treated group) versus placebo group.

FIG. 9. depicts the mean Systolic blood pressure vs actual time in schizophrenia patient infused with dexmedetomidine hydrochloride (treatment arm) and normal saline (placebo arm)—. Semi-log scale.

FIG. 10. depicts the mean Diastolic blood pressure vs actual time in schizophrenia patient infused with dexmedetomidine hydrochloride (treatment arm) and normal saline (placebo arm)—. Semi-log scale.

FIG. 11. depicts the mean heart rate vs actual time in schizophrenia patient infused with dexmedetomidine hydrochloride (treatment arm) and normal saline (placebo arm)—. Semi-log scale.

FIG. 12: depicts the maximum doses of dexmedetomidine hydrochloride received by schizophrenic patients for the treatment of agitation.

FIG. 13: depicts the total intravenous dose of dexmedetomidine hydrochloride received by schizophrenic patients for the treatment of agitation.

FIG. 14: depicts the mean Plasma concentration (pg/ml) vs actual time in schizophrenic patients infused with dexmedetomidine hydrochloride—Semi-log scale.

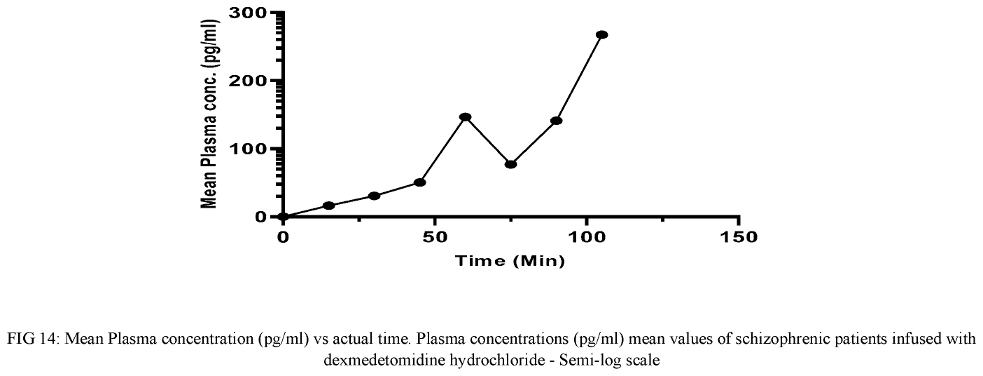

FIG. 15: depicts clinical opioid withdrawal symptoms during escalating infusion of dexmedetomidine hydrochloride (10 subjects) vs. placebo (5 subjects).

FIG. 16: depicts the mean Systolic blood pressure (mm of Hg) vs actual time in opioid withdrawal patients on Day 1 infused with dexmedetomidine hydrochloride and normal saline—Semi log scale.

FIG. 17: depicts the mean Systolic blood pressure (mm of Hg) vs actual time in opioid withdrawal patients on Day 2 infused with dexmedetomidine hydrochloride and normal saline—Semi log scale.

FIG. 18: depicts the mean Diastolic blood pressure (mm of Hg) vs actual time in opioid withdrawal patients on Day 1 infused with dexmedetomidine hydrochloride and normal saline—Semi log scale.

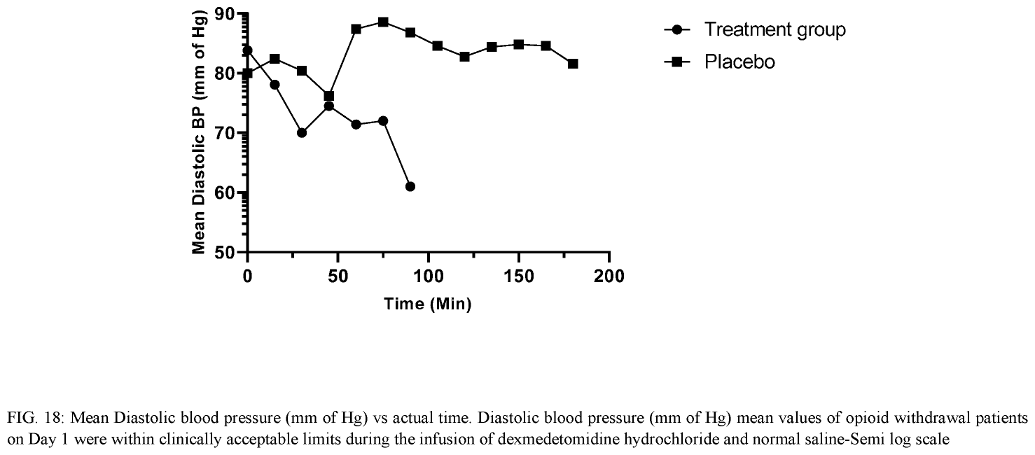

FIG. 19: depicts the mean Diastolic blood pressure (mm of Hg) vs actual time in opioid withdrawal patients on Day 2 infused with dexmedetomidine hydrochloride and normal saline—Semi log scale.

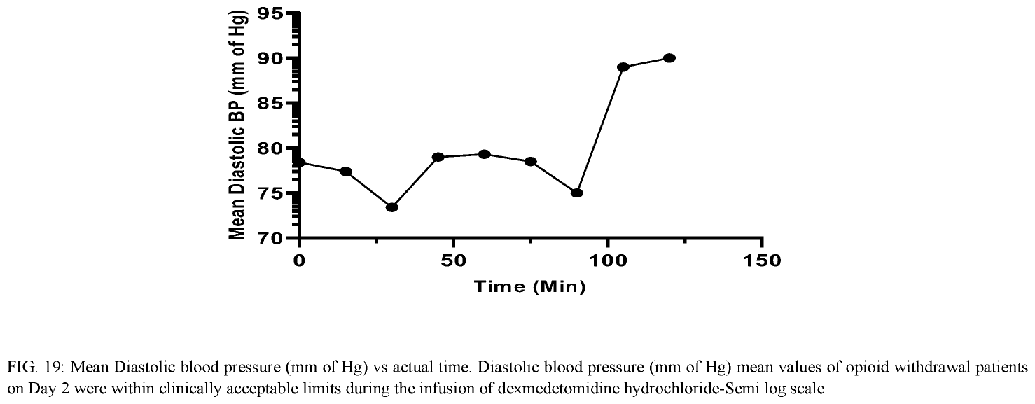

FIG. 20: depicts the mean heart rate (beats/min) vs actual time in opioid withdrawal patients on Day 1 infused with dexmedetomidine hydrochloride and normal saline—Semi log scale.

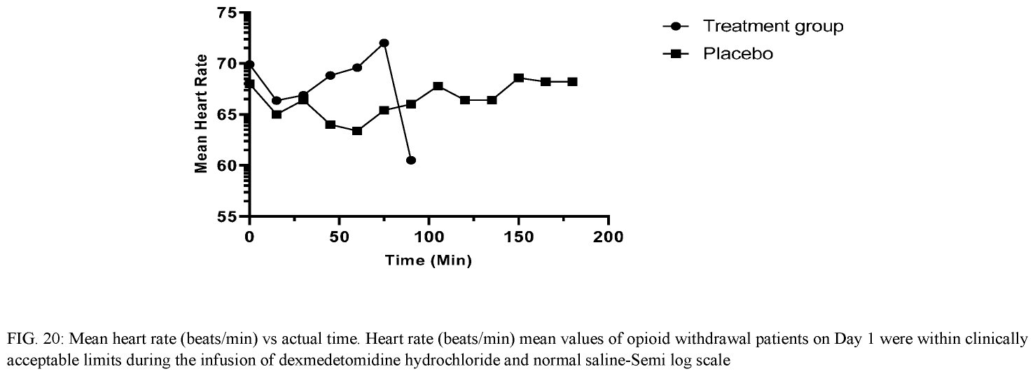

FIG. 21: depicts the mean heart rate (beats/min) vs actual time in opioid withdrawal patients on Day 2 infused with dexmedetomidine hydrochloride and normal saline—Semi log scale.

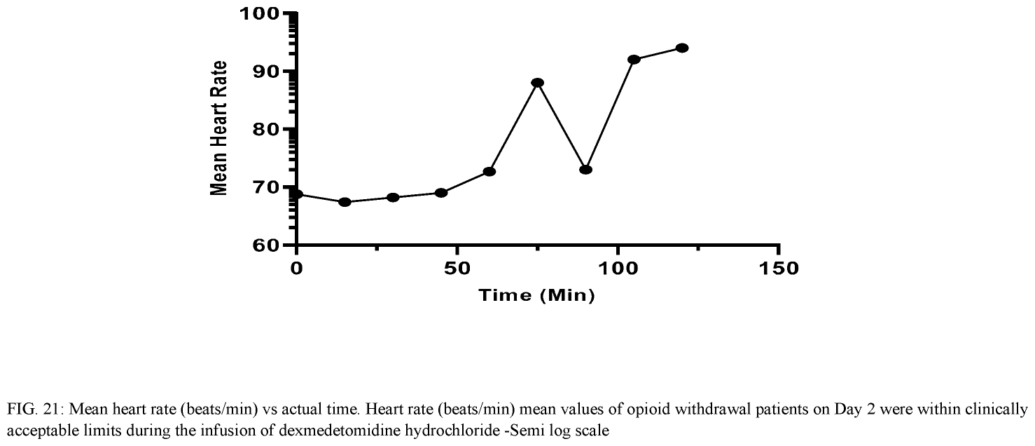

FIG. 22: depicts the mean Plasma concentration (pg/ml) vs actual time in opioid withdrawal patients on Day 1 infused with dexmedetomidine hydrochloride—Semi-log scale

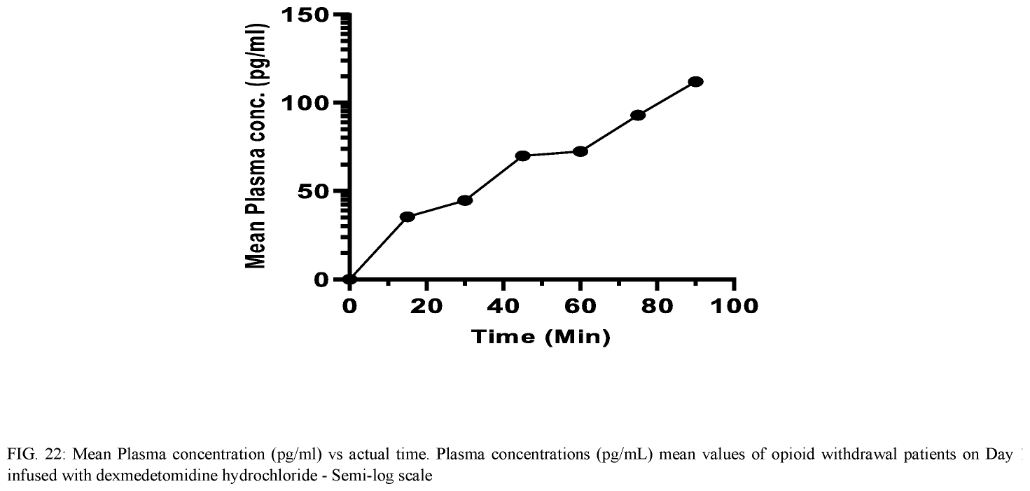

FIG. 23: depicts the mean Plasma concentration (pg/ml) vs actual time in opioid withdrawal patients on Day 2 infused with dexmedetomidine hydrochloride—Semi-log scale

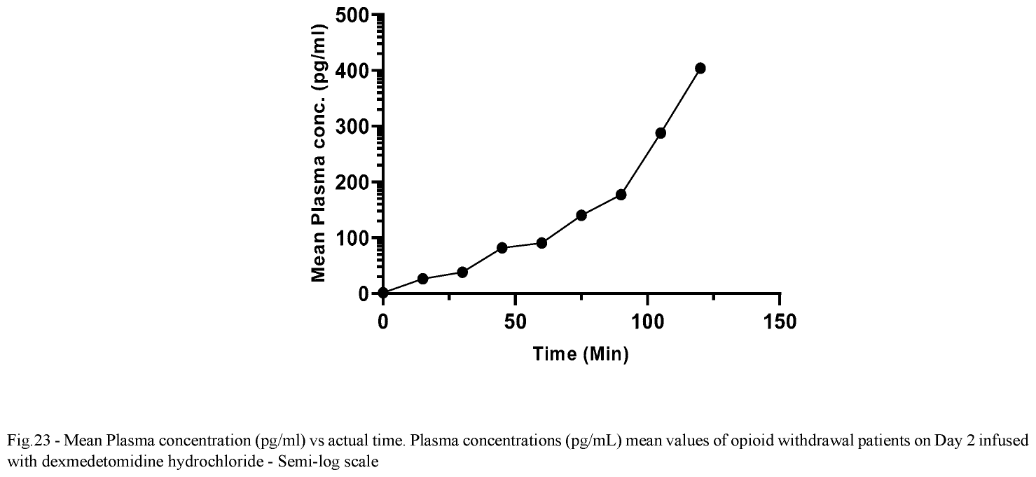

### DETAILED DESCRIPTION

### I. Abbreviation and Definition

AEs—Adverse events
BMI—Body mass index
BP—Blood pressure
CT—Computed tomography
COWS—Clinical Opiate Withdrawal Scale
CNS—Central nervous system
Dex—Dexmedetomidine
DBP—Diastolic blood pressure
ECG—Electrocardiogram
FSH—Follicular stimulating hormone
HCl—Hydrochloride
HR—Heart rate
Hr—Hours
IV—Intravenous
Kg—Kilogram
mcg/kg/hr—Mcg per kilograms per hour
μg/mL—Mcg per mililitre
mcg—Micrograms
mins—Minutes
mm of Hg—Millimeter of mercury
MRI—Magnetic Resonance Imaging
pg/mL—Picogram per milliliter
PTSD—post-traumatic stress disorder
PEC—Positive and Negative Symptom Scale—Excitatory Component
RASS—Richmond Agitation Sedation Scale
RS—Resolution time
SBP—Systolic blood pressure
SDAT—senile dementia of the Alzheimer type
SAEs—Serious adverse events
Yrs—Years

The terms “treating,” and “treatment,” as used herein refer to curative therapy, prophylactic therapy, and/or preventative therapy and can be used interchangeably.

As used herein, the term “about” refers to a value of plus or minus 10% of the indicated value.

As used herein, unless indicated otherwise, the terms “pharmaceutical composition”, “composition”, “formulation” are used interchangeably. Unless stated otherwise, the terms are meant to encompass, and are not limited to, pharmaceutical compositions containing drug substance, i.e. dexmedetomidine hydrochloride. The composition may also contain one or more “excipients” that are “inactive ingredients” or “compounds” devoid of pharmacological activity or other direct effect in the diagnosis, cure, mitigation, treatment, or prevention of disease or to affect the structure or any function of the human body.

As used herein, the term “an effective amount” is interchangeable with “therapeutically effective dose,” or “therapeutically effective amount,” and refers to an amount sufficient to produce the desired effect. An effective amount is sufficient to cause an improvement in a clinically significant condition of the subject.

As used herein, the term “subject” refers to a human patient.

The term “agitation”, as used herein, means irritability, emotional outburst, impaired thinking, or excess motor and verbal activity that may occur due to either dysfunction of specific brain regions such as frontal lobes or due to dysfunction of neurotransmitter systems such as dopamine and nor-epinephrine. In the present disclosure, agitation also includes aggression and hyper-arousal in post-traumatic stress disorder. The agitation may be acute or chronic.

The term “the signs of agitation” includes excessive motor activity (examples include: pacing, rocking, gesturing, pointing fingers, restlessness, performing repetitious mannerisms), verbal aggression (e.g. yelling, speaking in an excessively loud voice, using profanity, screaming, shouting, threatening other people), physical aggression (e.g. grabbing, shoving, pushing, clenching hands into fists, resisting, hitting others, kicking objects or people, scratching, biting, throwing objects, hitting self, slamming doors, tearing things, and destroying property)

The term “acute agitation” means agitation that occurs rapidly and is severe and sudden in onset. Acute agitation may be associated with, for example, neurodegenerative disease, Psychiatric disorders, or drug withdrawal although it may particularly exist in psychiatric conditions.

The term “neurodegenerative disease” includes, but is not limited to, Alzheimer's disease, senile dementia of the Alzheimer type (SDAT), frontotemporal dementia (or Pick's disease), dementia, dementia with Lewy bodies, posttraumatic stress disorder, Parkinson's disease, vascular dementia, vascular cognitive impairment, Huntington's disease, multiple sclerosis, Creutzfeldt-Jakob disease, multiple system atrophy, progressive supranuclear palsy or other related neurodegenerative diseases.

The term “neuropsychiatric conditions” includes, but is not limited to, schizophrenia, bipolar illness (bipolar disorder, bipolar mania), depression, delirium or other related neuropsychiatric conditions. The term “hyper-arousal” is withdrawal symptom and a specific cluster of symptoms associated with post-traumatic stress disorder (PTSD).

### II. Pharmaceutical Agent

In one embodiment, the present disclosure employs dexmedetomidine (also known (+)-4-(S)-(2,3-dimethylphenyl)ethyl-1H-imidazole) in the form of the monohydrochloride salt. Dexmedetomidine is a new generation highly selective α2-adrenergic receptor (α2-AR) agonist. Dexmedetomidine hydrochloride has been approved by the FDA as an IV infusion product (Precedex®) for the indication of sedation in certain subjects.

### III. Methods

The following are various aspects of the methods described in the present disclosure:

1. A method for treating agitation (e.g. acute agitation) or signs of agitation in a human subject who is alert prior to treatment, comprising administering dexmedetomidine hydrochloride intravenously to the subject at a cumulative dose of about 0.1 mcg/kg to about 1 mcg/kg to achieve the desired anti-agitation effect.
2. A method for treating agitation (e.g. acute agitation) or signs of agitation in a human subject who is alert prior to treatment, comprising administering dexmedetomidine hydrochloride intravenously to the subject to a total dose in a range from about 2 mcg to about 100 mcg to achieve the desired anti-agitation effect.
3. A method for treating agitation (e.g. acute agitation) or signs of agitation in a human subject who is alert prior to treatment, comprising administering dexmedetomidine hydrochloride intravenously to the subject at a cumulative dose of about 0.1 mcg/kg to about 1 mcg/kg to achieve the desired anti-agitation effect, wherein the desired anti-agitation effect is achieved without also producing clinically meaningful effects on blood pressure and/or heart rate.
4. A method for treating agitation (e.g. acute agitation) or signs of agitation in a human subject who is alert prior to treatment, comprising administering dexmedetomidine hydrochloride intravenously to the subject to a total dose in a range from about 2 mcg to about 100 mcg to achieve the desired anti-agitation effect, wherein the desired anti-agitation effect is achieved without also producing clinically meaningful effects on blood pressure and/or heart rate.
5. A method of treating agitation (e.g. acute agitation) or signs of agitation in a human subject suffering from senile dementia of the Alzheimer's type (SDAT) who is alert prior to treatment, comprising administering dexmedetomidine hydrochloride intravenously to the subject at a cumulative dose of about 0.15 mcg/kg to about 0.75 mcg/kg to achieve the desired anti-agitation effect, which effect persists for up to about 30 minutes after discontinuation of administration of dexmedetomidine hydrochloride, and wherein the reduction in agitation is associated with the attainment of a mild sedative effect.
6. A method of treating agitation (e.g. acute agitation) or signs of agitation in a human subject suffering from senile dementia of the Alzheimer's type (SDAT) who is alert prior to treatment, comprising administering dexmedetomidine hydrochloride intravenously to the subject at a total dose of about 5 mcg to about 40 mcg to achieve the desired anti-agitation effect, which effect persists for up to about 30 minutes after discontinuation of administration of dexmedetomidine hydrochloride, and wherein the reduction in agitation is associated with the attainment of a mild sedative effect.
7. A method of treating agitation (e.g. acute agitation) or signs of agitation in a human subject suffering from schizophrenia who is alert prior to treatment, comprising administering dexmedetomidine hydrochloride intravenously to the subject at a total dose of about 5 mcg to about 40 mcg to achieve the desired anti-agitation effect, wherein agitation is reduced to a value of 7 or below 7 according to the PEC scale, and wherein the reduction in agitation is associated with the attainment of a mild sedative effect.
8. A method of treating agitation (e.g. acute agitation) or signs of agitation in a human subject suffering from opioid withdrawal disorder who is alert prior to treatment, comprising administering dexmedetomidine hydrochloride intravenously to the subject at a total dose of about 3 mcg to about 90 mcg to achieve at least a 50% reduction in COW score.
9. A method of treating agitation (e.g. acute agitation) or signs of agitation in a human subject suffering from senile dementia of the Alzheimer's type (SDAT) who is alert prior to treatment, comprising administering to the subject dexmedetomidine hydrochloride intravenously by continuous infusion at an infusion rate up to about 0.5 mcg/kg/hr to achieve a maximum plasma concentration (Cₘₐₓ) of up to about 400 pg/ml, together with the attainment of a mild sedative effect.
10. A method of treating agitation (e.g. acute agitation) or signs of agitation in a human subject suffering from schizophrenia who is alert prior to treatment, comprising administering to the subject dexmedetomidine hydrochloride intravenously by continuous infusion at an infusion rate up to about 0.5 mcg/kg/hr to achieve a maximum plasma concentration (Cₘₐₓ) of up to about 410 pg/ml, wherein agitation is reduced to a value of 7 or below 7 according to the PEC scale, and wherein the reduction in agitation is associated with the attainment of a mild sedative effect.
11. A method of treating agitation (e.g. acute agitation) or signs of agitation in a human subject suffering from opioid withdrawal disorder who is alert prior to treatment, comprising administering to the subject dexmedetomidine hydrochloride intravenously by continuous infusion at an infusion rate up to about 0.7 mcg/kg/hr to achieve a maximum plasma concentration (Cₘₐₓ) of up to about 410 pg/ml, and at least a 50% reduction in COW score.

In one aspect of the disclosure, a method is disclosed herein comprising administering dexmedetomidine hydrochloride by intravenous infusion to an alert, agitated, subject over a period of about 20 minutes to about 180 minutes to reduce the RASS score to −1.

In a particular aspect, the subject is dosed at about 0.1 mcg/kg/hr to about 0.7 mcg/kg/hr.

In a further particular aspect the dosing is initiated at about 0.1 mcg/kg/hr, and increased in about 0.1 mcg/kg/hr increments every 30 minutes until a RASS score to −1 is achieved. In another particular aspect, the RASS score of −1 is maintained for about 20 minutes to about 120 minutes following cessation of treatment.

In one embodiment of the disclosure, a method is disclosed herein for optimizing the blood levels of dexmedetomidine in an alert, agitated, subject, the method comprising administering dexmedetomidine hydrochloride by intravenous infusion to the subject at an initial dose (e.g. about 0.1 mcg/kg/hr or about 0.2 mcg/kg/hr), measuring the amount of dexmedetomidine in the plasma at regular intervals (e.g. every 15 minutes), and adjusting the dose as necessary to achieve plasma levels of dexmedetomidine in the range of about 10 pg/ml to about 460 pg/ml.

The disclosure also provides methods of treating agitation in an alert subject, comprising administering dexmedetomidine hydrochloride by continuous intravenous infusion at an initial dose followed by one or more different doses until a reduction in agitation is achieved, e.g. an initial dose of about 0.1 mcg/kg/hr or about 0.2 mcg/kg/hr followed by increasing doses at about 0.1 mcg/kg/hr increments. The initial dose may also constitute an initial loading dose with subsequent one or more doses being administered at a lower rate than the loading dose until a reduction in agitation is achieved.

It will be appreciated that, in certain alert, agitated, subjects a single dose will achieve a reduction in agitation, whereas in other subjects a second, or third, or fourth, or fifth, or sixth etc. dose, which will be different from the preceding dose (e.g. an ascending dosage regimen), may be required to achieve a reduction in agitation.

In certain embodiments where a second dose is required to achieve a reduction in agitation in the subject, the dose may be administered for about 0.5 hours to about 3 hours, preferably about 0.5 hours to about 1.5 hours.

In certain embodiments, the continuous IV infusion is administered at the first rate for about 0.5 hours to about 3 hours.

In certain embodiments, the continuous IV infusion is administered at the first rate for about 0.5 hours to about 2 hours.

In certain embodiments, the continuous IV infusion is administered at the first rate for about 0.5 hours.

In certain embodiments, the total time of the continuous IV infusion at the first rate plus the time of the infusion at the second rate is about 3 hours, preferably about 1 hour.

In certain embodiments, the total time of the continuous IV infusion at the first rate plus the time of the infusion more than one subsequent rates are about 3 hours, preferably about 2.5 hours.

In certain embodiments, the continuous IV infusion is administered once per week. In certain embodiments, the continuous IV infusion is administered for two or more consecutive weeks, for three or more consecutive weeks, for four or more consecutive weeks, for five or more consecutive weeks, for six or more consecutive weeks, for seven or more consecutive weeks, or for eight or more consecutive weeks.

### IV. Reduction in Agitation

The present disclosure provides a dosage regimen, wherein the administration of dexmedetomidine hydrochloride intravenously to alert, agitated, subjects is able to achieve mild sedation (i.e. a RASS score of −1) in subjects at a dexmedetomidine exposure level that does not produce clinically meaningful effects on blood pressure and/or heart rate. The mild sedative effect may persist for up to about 2 hours, e.g. about 1.5 to 2 hours, a clinically relevant duration. In one aspect of the present disclosure, the IV dose of dexmedetomidine hydrochloride is adjusted, e.g. titrated, to account for inter-individual variability to response to dexmedetomidine hydrochloride.

The level of agitation in subjects may be assessed herein using the PEC (Positive and Negative Symptom Scale—Excitatory Component) scale, which is a known five item scale that measures symptoms of agitation, namely excitement, tension, hostility, uncooperativeness, and poor impulse control, with each item rated from 1 (absent) to 7 (extremely severe). Combined scores range from 5 to 35. A score of 20 or more corresponds to severe agitation.

The present disclosure provides a dosage regimen, wherein the administration of dexmedetomidine hydrochloride intravenously to alert, agitated, subjects is able to achieve a PEC score of 7 or below (i.e. negligible agitation) in subjects at a dexmedetomidine exposure level that does not produce clinically meaningful effects on blood pressure and/or heart rate.

The level of agitation or hyper-arousal in subjects treated for opioid abuse, along with other measurements of symptoms of opioid withdrawal, may be assessed using the COW scale. Advantageously, a 50% reduction in COW score was achieved in 90 minutes in all subjects administered dexmedetomidine hydrochloride intravenously according to the dosage regimen herein.

Thus, the present disclosure provides a dosage regimen, wherein the administration of dexmedetomidine hydrochloride intravenously to subjects agitated as a result of opioid withdrawal symptoms is able to achieve a 50% reduction in COW score, for example within about 90 minutes.

### V. Administration and Dosages

The present disclosure provides for the administration of dexmedetomidine hydrochloride by the intravenous (IV) route. In one embodiment, the administration is by continuous IV infusion. In another embodiment, the administration is by one or more IV injections. In a further embodiment, the administration is by a combination of one or more IV injections and continuous IV infusion, for example, one or more initial IV injections followed by continuous IV infusion for a period of time or an initial continuous IV infusion for a period of time followed by one or more IV injections. The appropriate regimen according to the present disclosure is designed to the individual alert, agitated, subject to provide a reduction in agitation, preferably without inducing significant sedation and/or without producing clinically meaningful effects on blood pressure and/or heart rate. As defined herein, significant sedation means a RASS score of −3 to −5. Preferably, the dosage regimen of the present disclosure is designed to provide a RASS score of −1.

In one particular aspect, dexmedetomidine hydrochloride is administered intravenously to an alert, agitated, subject at a cumulative dose of about 0.1 mcg/kg to about 1 mcg/kg to achieve the desired anti-agitation effect. Preferably, this cumulative dose achieves the desired anti-agitation effect without inducing significant sedation and/or without producing clinically meaningful effects on blood pressure and/or heart rate. In a particularly preferred embodiment, this cumulative dose achieves the desired anti-agitation effect without producing clinically meaningful effects on blood pressure and/or heart rate whilst providing a RASS score of −1.

In another particular aspect, dexmedetomidine hydrochloride is administered intravenously to an alert, agitated, subject to a total dose in a range from about 2 mcg to about 100 mcg to achieve the desired anti-agitation effect. Preferably, this total dose achieves the desired anti-agitation effect without inducing significant sedation and/or without producing clinically meaningful effects on blood pressure and/or heart rate. In a particularly preferred embodiment, this total dose achieves the desired anti-agitation effect without producing clinically meaningful effects on blood pressure and/or heart rate whilst providing a RASS score of −1.

In further particular aspects, dexmedetomidine hydrochloride may be administered by continuous IV infusion over a period of about 20 minutes, about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes, 150 minutes, about 160 minutes, about 170 minutes or about 180 minutes. Thus, the continuous IV infusion may be administered in a range of time over about 20 minutes to about 180 minutes,

In one aspect, the dosage administered by continuous IV infusion (i.e. the infusion rate), may be about 0.1 mcg/kg/hr to about 0.7 mcg/kg/hr, for example, about 0.1 mcg/kg/hr, about 0.2 mcg/kg/hr, about 0.3 mcg/kg/hr, about 0.4 mcg/kg/hr, about 0.5 mcg/kg/hr, about 0.6 mcg/kg/hr or about 0.7 mcg/kg/hr.

In a particular aspect, dexmedetomidine hydrochloride is administered by continuous IV infusion at a rate of about 0.1 mcg/kg/hr to about 0.7 mcg/kg/hr, for example, about 0.1 mcg/kg/hr, about 0.2 mcg/kg/hr, about 0.3 mcg/kg/hr, about 0.4 mcg/kg/hr, about 0.5 mcg/kg/hr, about 0.6 mcg/kg/hr or about 0.7 mcg/kg/hr, over a period of about 20 minutes to about 180 minutes.

The infusion rate may remain constant during the period of infusion or may be modified, e.g. escalated, to achieve the appropriate dose at which agitation is reduced to acceptable levels according to the relevant scoring system used, e.g. RASS, PEC and/or COWS.

In particular aspects, the initial dose of dexmedetomidine hydrochloride is about 0.1 mcg/kg/hr and dose escalation occurred every 30 minutes by increasing the infusion rate by about 0.1 mcg/kg/hr to a maximum infusion of about 0.6 mcg/kg/hr or about 0.7 mcg/kg/hr.

In particular aspects, the initial dose of dexmedetomidine hydrochloride is about 0.2 mcg/kg/hr and dose escalation occurred every 30 minutes by increasing the infusion rate by about 0.1 mcg/kg/hr to a maximum infusion of about 0.6 mcg/kg/hr.

The continuous IV infusion may be performed one or more times (i.e., for one or more cycles), with or without intervening rest periods. Similarly, the continuous infusion may be performed two or more times (i.e., for two or more cycles), with or without intervening rest periods, preferably without intervening rest periods. Each cycle may be the same or different. For example, if there are two cycles, they may, independently, have the same or different duration, the same or different dosage, etc. In one embodiment, the continuous IV infusion is performed for two or more cycles, for example from 2 to 3 cycles, from 2 to 4 cycles, from 2 to 5 cycles, etc., with or without intervening rest periods. In one embodiment, the continuous IV infusion is performed for three or more cycles, for example from 3 to 4 cycles, from 3 to 5 cycles, etc., with or without intervening rest periods, preferably without intervening rest periods. In one embodiment, if the continuous IV infusion is performed for two or more cycles, without intervening rest period, for example, from 30 minutes to 3 hours, from about 1 hour to 2.5 hours etc.

In a preferred aspect, the dexmedetomidine hydrochloride infusion rate is from about 0.1 to about 0.6 mcg/kg/hr, preferably about 0.2 to about 0.5 mcg/kg/hr.

In a further preferred aspect, the mean dexmedetomidine hydrochloride infusion rate is about 0.34 mcg/kg/hr when administered to Alzheimer's subjects.

In a further preferred aspect, the mean dexmedetomidine hydrochloride infusion rate is about 0.29 mcg/kg/hr when administered to opioid withdrawal subjects.

In a further preferred aspect, the mean dexmedetomidine hydrochloride infusion rate is about 0.36 mcg/kg/hr when administered to schizophrenic subjects.

In another aspect, the cumulative dose administered to treat an alert, agitated, subject may be from about 0.1 mcg/kg to about 1 mcg/kg. For example, the dosage administered during this period may be about 0.1 mcg/kg to about 0.8 mcg/kg, including about 0.15 mcg/kg to about 0.75 mcg/kg; for example, about 0.1 mcg/kg, about 0.15 mcg/kg, about 0.2 mcg/kg, about 0.25 mcg/kg, about 0.3 mcg/kg, about 0.3.5 mcg/kg, about 0.4 mcg/kg, about 0.45 mcg/kg, about 0.5 mcg/kg, about 0.55 mcg/kg, about 0.6 mcg/kg, about 0.65 mcg/kg, about 0.7 mcg/kg, about 0.75 mcg/kg or about 0.8 mcg/kg. In a preferred embodiment, the cumulative dose administered during the period may be about from about 0.15 mcg/kg to about 0.40 mcg/kg. In another preferred embodiment, the dosage administered during this period may be about 0.1 mcg/kg to about 0.3 mcg/kg; for example, about 0.1 mcg/kg, about 0.2 mcg/kg, or about 0.3 mcg/kg.

In particular aspects, the total dose administered to treat a single agitation episode an alert, agitated, subject may be in a range of about 2 mcg to about 150 mcg, 2 mcg to about 100 mcg, about 2 mcg to about 90 mcg, about 3 mcg to about 90 mcg, about 2 mcg to about 85 mcg, about 2 mcg to about 80 mcg, about 2 mcg to about 75 mcg, about 2 mcg to about 70 mcg, about 2 mcg to about 65 mcg, about 2 mcg to about 60 mcg, about 2 mcg to about 40 mcg, about 5 mcg to about 40 mcg, about 10 mcg to about 60 mcg, about 10 mcg to about 50 mcg, about 20 mcg to about 50 mcg, or about 20 mcg to about 40 mcg.

In specific embodiments, the total dose to treat a single agitation episode an alert, agitated, subject may be in a range of about 2 mcg to about 90 mcg, about 2 mcg to about 85 mcg, about 2 mcg to about 80 mcg, about 2 mcg to about 75 mcg, about 2 mcg to about 70 mcg, about 2 mcg to about 65 mcg, about 2 mcg to about 60 mcg, about 2 mcg to about 40 mcg, about 3 mcg to about 85 mcg, about 4 mcg to about 85 mcg, about 5 mcg to about 85 mcg, about 6 mcg to about 85 mcg, about 7 mcg to about 85 mcg, about 10 mcg to about 85 mcg, about 15 mcg to about 85 mcg; about 20 mcg to about 85 mcg; about 25 mcg to about 85 mcg, about 30 mcg to about 85 mcg, about 35 mcg to about 85 mcg; about 40 mcg to about 85 mcg, about 45 mcg to about 85 mcg, about 50 mcg to about 85 mcg; about 5 mcg to about 70 mcg; about 10 mcg to about 70 mcg, about 15 mcg to about 70 mcg about; about 20 mcg to about 70 mcg; about 25 mcg to about 70 mcg; about 30 mcg to about 70 mcg; about 35 mcg to about 70 mcg; about 40 mcg to about 70 mcg; about 45 mcg to about 70 mcg; about 50 mcg to about 70 mcg; about 10 mcg to about 60 mcg; 15 mcg to about 60 mcg, about 20 mcg to about 60 mcg, about 25 mcg to about 60 mcg; about 30 mcg to about 60 mcg; about 35 mcg to about 60 mcg; about 40 mcg to about 60 mcg; about 45 mcg to about 60 mcg; about 50 mcg to about 60 mcg; about 10 mcg to about 50 mcg; about 15 mcg to about 50 mcg; about 20 mcg to about 50 mcg, about 25 mcg to about 50 mcg; about 30 mcg to about 50 mcg, about 35 mcg to about 50 mcg; about 40 mcg to about 50 mcg; about 5 mcg to about 40 mcg, about 10 mcg to about 40 mcg, about 15 mcg to about 40 mcg, about 20 mcg to about 40 mcg, about 25 mcg to about 40 mcg, about 30 mcg to about 40 mcg, and about 35 mcg to about 40 mcg.

In particular aspects, the total dose may be about 3 mcg, about 4 mcg, about 5 mcg, about 6 mcg, about 7 mcg, about 8 mcg, about 9 mcg, about 10 mcg, about 11 mcg, about 12 mcg, about 13 mcg, about 14 mcg, about 15 mcg, about 16 mcg, about 17 mcg, about 18 mcg, about 19 mcg, about 20 mcg, about 21 mcg, about 22 mcg, about 23 mcg, about 24 mcg, about 25 mcg, about 26 mcg, about 27 mcg, about 28 mcg, about 29 mcg, about 30 mcg, about 31 mcg, about 32 mcg, about 33 mcg, about 34 mcg, about 35 mcg, about 36 mcg, about 37 mcg, about 38 mcg, about 39 mcg, about 40 mcg, about 41 mcg, about 42 mcg, about 43 mcg, about 44 mcg, about 45 mcg, about 46 mcg, about 47 mcg, about 48 mcg, about 49 mcg, about 50 mcg, about 55 mcg, about 60 mcg, about 65 mcg, about 70 mcg, about 75 mcg, about 80 mcg, about 85 mcg or about 90 mcg.

In one specific embodiment, the total dose to treat a single agitation episode in an SDAT subject may be about 5 mcg, about 6 mcg, about 7 mcg, about 8 mcg, about 9 mcg, about 10 mcg, about 11 mcg, about 12 mcg, about 13 mcg, about 14 mcg, about 15 mcg, about 16 mcg, about 17 mcg, about 18 mcg, about 19 mcg, about 20 mcg, about 21 mcg, about 22 mcg, about 23 mcg, about 24 mcg, about 25 mcg, about 26 mcg, about 27 mcg, about 28 mcg, about 29 mcg, about 30 mcg, about 31 mcg, about 32 mcg, about 33 mcg, about 34 mcg, about 35 mcg, about 36 mcg, about 37 mcg, about 38 mcg, about 39 mcg or about 40 mcg,

In another specific embodiment, the total dose to treat a single agitation episode in a schizophrenic subject may be about 5 mcg, about 6 mcg, about 7 mcg, about 8 mcg, about 9 mcg, about 10 mcg, about 11 mcg, about 12 mcg, about 13 mcg, about 14 mcg, about 15 mcg, about 16 mcg, about 17 mcg, about 18 mcg, about 19 mcg, about 20 mcg, about 21 mcg, about 22 mcg, about 23 mcg, about 24 mcg, about 25 mcg, about 26 mcg, about 27 mcg, about 28 mcg, about 29 mcg, about 30 mcg, about 31 mcg, about 32 mcg, about 33 mcg, about 34 mcg, about 35 mcg, about 36 mcg, about 37 mcg, about 38 mcg, about 39 mcg or about 40 mcg,

In another specific embodiment, the total dose to provide at least a 50% reduction in COWS score may be about 3 mcg, about 4 mcg, about 5 mcg, about 6 mcg, about 7 mcg, about 8 mcg, about 9 mcg, about 10 mcg, about 11 mcg, about 12 mcg, about 13 mcg, about 14 mcg, about 15 mcg, about 16 mcg, about 17 mcg, about 18 mcg, about 19 mcg, about 20 mcg, about 21 mcg, about 22 mcg, about 23 mcg, about 24 mcg, about 25 mcg, about 26 mcg, about 27 mcg, about 28 mcg, about 29 mcg, about 30 mcg, about 31 mcg, about 32 mcg, about 33 mcg, about 34 mcg, about 35 mcg, about 36 mcg, about 37 mcg, about 38 mcg, about 39 mcg, about 40 mcg, about 41 mcg, about 42 mcg, about 43 mcg, about 44 mcg, about 45 mcg, about 46 mcg, about 47 mcg, about 48 mcg, about 49 mcg, about 50 mcg, about 55 mcg, about 60 mcg, about 65 mcg, about 70 mcg, about 75 mcg, about 80 mcg or about 85 mcg.

In particular aspects, the desired human plasma concentrations of dexmedetomidine to provide relief from agitation in an alert subject may range from about 10 pg/ml to about 450 pg/ml; for example, about 11 pg/ml to about 404 pg/ml, about 20 pg/ml to about 404 pg/ml, about 40 pg/ml to about 404 pg/ml, about 60 pg/ml to about 404 pg/ml, about 80 pg/ml to about 404 pg/ml, about 100 pg/ml to about 404 pg/ml, about 150 pg/ml to about 404 pg/ml, about 200 pg/ml to about 404 pg/ml, about 250 pg/ml to about 404 pg/ml, about 300 pg/ml to about 404 pg/ml, about 50 pg/ml to about 370 pg/ml, about 100 pg/ml to about 370 pg/ml, about 150 pg/ml to about 370 pg/ml, about 200 pg/ml to about 370 pg/ml, about 250 pg/ml to about 370 pg/ml, about 100 pg/ml to about 340 pg/ml, about 150 pg/ml to about 340 pg/ml, about 180 pg/ml to about 340 pg/ml, about 200 pg/ml to about 340 pg/ml, about 20 pg/ml to about 250 pg/ml, about 50 pg/ml to about 250 pg/ml, about 100 pg/ml to about 250 pg/ml, or about 150 pg/ml to about 250 pg/ml.

In a particular embodiment, the desired human plasma concentrations of dexmedetomidine to provide relief from agitation in an alert subject suffering from Alzheimer's disease (e.g. SDAT) may be about 10 pg/ml to about 400 pg/ml; for example, about 60 pg/ml to about 380 pg/ml, about 80 pg/ml to about 380 pg/ml, about 100 pg/mL to about 380 pg/ml, or about 102 pg/ml to about 380 pg/ml, preferably about 102 pg/ml to about 380 pg/mL.

In another particular embodiment, the desired human plasma concentrations of dexmedetomidine to provide relief from agitation in an alert subject suffering from opiate withdrawal symptoms may be about 10 pg/ml to 410 pg/ml for example, about 22 pg/ml to 406 pg/ml, about 23.28 pg/ml to about 403.80 pg/ml or about 11.33 pg/mL to about 168.47 pg/ml.

In another particular embodiment, the desired human plasma concentrations of dexmedetomidine to provide relief from agitation in an alert subject suffering from schizophrenia may be about 10 pg/ml to 410 pg/ml for example, about 20 pg/mL to about 406 pg/mL, about 22 pg/mL to 406 pg/ml or about 15 pg/ml to about 270 pg/ml.

In particular aspects, the time to reach maximum plasma concentration is about 15 minutes to about 150 minutes, more particularly about 60 minutes to about 150 minutes.

In one particular embodiment, the time to reach maximum plasma concentration in a subject suffering from Alzheimer's disease (e.g. SDAT) may be about 15 minutes to about 150 minutes, for example, from about 60 minutes to about 140 minutes, about 60 minutes to about 135 minutes. In particular aspects, the time to reach maximum plasma concentration in a healthy subject may be about 30 minutes to about 160 minutes, for example, from about 30 minutes to about 150 minutes, about 30 minutes to about 90 minutes, about 90 minutes to about 150 minutes.

In another particular embodiment, the time to reach maximum plasma concentration in a subject suffering from schizophrenia may be about 15 minutes to 120 minutes, for example, from about 15 minutes to about 105 minutes, from about 15 minutes to about 90 minutes, about 30 minutes to about 105 minutes, about 30 minutes to about 90 minutes.

In yet another particular embodiment, the time to reach maximum plasma concentration in a subject suffering from opiate withdrawal patients may be about 15 minutes to about 150 minutes, for example, from about 15 minutes to about 105 minutes, from about 15 minutes to about 90 minutes, from about 30 minutes to about 120 minutes, from about 30 minutes to about 90 minutes.

### VI. Pharmaceutical Compositions

The present disclosure provides dexmedetomidine hydrochloride for administration by the intravenous route, as a single bolus injection, as multiple injections or as a continuous IV infusion. Formulations suitable for administering dexmedetomidine hydrochloride by injection or continuous infusion and methods for their preparation are within the common general knowledge of the skilled artisan. Published procedures may also be consulted, for example as described in in Avis et al., 1992. Specifically, for continuous IV infusions, the Precedex® literature, including FDA submissions, may be consulted.

Dexmedetomidine hydrochloride may be presented, for example, in unit dose form in ampoules, pre-filled syringes, small volume infusion containers, or multi-dose containers optionally with an added preservative. The dexmedetomidine hydrochloride formulations may take such forms as a solution (e.g., aqueous, non-aqueous), a suspension (e.g., aqueous, non-aqueous), or an emulsion (e.g., oil-in-water, water-in-oil), and may contain formulation agents such as suspending agents, stabilizing agents, dispersing agents, carriers, diluents, excipients, adjuvants, buffers, preservatives, anti-oxidants, stabilizers, solubilizers, surfactants (e.g., wetting agents), etc. Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulation may be prepared by any methods well known in the art of pharmacy. Pharmaceutical compositions for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Formulations suitable for parenteral administration (e.g., by injection), include aqueous or non-aqueous, isotonic, pyrogen-free, sterile liquids (e.g., solutions, suspensions), in which the dexmedetomidine is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, vehicle, co-solvent, tonicity adjuster, solubilizing agents and solutes which render the formulation isotonic with the blood (or other relevant bodily fluid) of the intended recipient. The vehicle used for preparation of composition is water, a saline solution, another aqueous liquid or non-aqueous liquid. The liquid vehicle can be a solvent of dispersion medium containing, aqueous vehicle used for formulation is water, water for injection, sodium chloride injection, ringer's solution, lactated ringer's injection and sterile water for injection. Nonaqueous solvents can also be used in formulation development, they may be organic solvent such as ethanol, polyol (e.g., glycerol, propylene glycol, and liquid polyethylene glycol, and the like) or oily vehicles such as castor oil, arachis oil, sesame oil or mixtures thereof. Solubilizing agents added in injection formulation to increase dissolution of drug. Solubilizing agents can be polyoxyethylene castor oil, alpha and gamma cyclodextrins, cationic surfactants, anionic surfactants, nonionic surfactants or mixtures thereof. Cosolvent used in the formulation to increase the solubility of drug or to reduce the viscosity of oily vehicle, such as castor oil, arachis oil, sesame oil. Co-solvent can be ethanol, glycerol, isopropyl alcohol, surfactants or mixtures thereof. Microorganism growth increases under ordinary conditions of storage and use, for prevention of microorganism growth preservative also added in these preparations. The prevention of the action of microorganisms can be brought about by various antibacterial and antifungal agents, for example, parabens, chlorobutanol, phenol, sorbic acid, thimerosal, or mixtures thereof. Preservatives also help against contaminating action of bacteria and fungi during manufacturing and storage of formulation. Isotonic agents added to adjust tonicity of injectable. Suitable isotonic agents example including sugars, phosphate buffers, sodium chloride, or mixtures thereof. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

The parenteral formulation can be sterilized. Non-limiting examples of sterilization techniques include filtration through a bacterial-retaining filter, terminal sterilization, incorporation of sterilizing agents, irradiation, heating, vacuum drying, and freeze drying.

Typically, parenteral formulations (i.e., formulations suitable for parenteral administration, e.g., intravenous infusion) are typically packaged in plastic or glass large volume parenteral (LVP) containers to which is attached a suitable intravenous (i.v.) set at the time of infusion. Venous entry is typically by a metal needle or plastic catheter.

In certain embodiments, the dexmedetomidine liquid composition comprises dexmedetomidine hydrochloride at a concentration of about 0.5 μg/mL, about 1.0 μg/mL, about 2.0 μg/mL, about 4.0 μg/ml, or about 5 μg/ml, preferably about 4.0 μg/ml.

The infusion may be infused according to a continuous or intermittent dose schedule. A continuous schedule typically involves the non-stop infusion of a relatively large volume of fluid (e.g., 1 litre per 8-hour period for adults). Continuous therapy typically additionally provides fluid, electrolytes, agents to adjust acid-base balance, nutrients, and some other drugs. The total fluid intake must not exceed the patient's requirements (approximately 2400 ml per day for an adult).

The dexmedetomidine hydrochloride composition may be provided as a concentrate which is diluted prior to administration to the subject. Alternatively, a premixed composition may be used, for example, as described in International Patent Application Publication No. WO2013103378, the entire contents of which are expressly incorporated herein by reference.

### VII. Conditions Treated

In one embodiment, the disease or disorder is an agitation or signs of agitation associated with a neurodegenerative disease.

In another embodiment, the disease or disorder is an agitation or signs of agitation associated with a neuropsychiatric disease.

In a specific embodiment, the neurodegenerative disease is selected from Alzheimer's disease, senile dementia of the Alzheimer's type (SDAT), fronto-temporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, and progressive supranuclear palsy.

In another specific embodiment, the neuropsychiatric disease is selected from schizophrenia, bipolar disorder, bipolar mania, other bipolar illness, depression, and delirium.

In a further embodiment, the disease or disorder is an agitation or signs of agitation associated with alcohol, opioid or substance abuse withdrawal. In a particular embodiment, agitation is associated with opioid withdrawal.

### VIII. Kits

One aspect of the present disclosure pertains to a kit comprising (a) a dexmedetomidine hydrochloride composition, preferably provided in a suitable container and/or with suitable packaging; and (b) instructions for use, e.g., written instructions on how to administer the dexmedetomidine hydrochloride composition in accordance with the present disclosure, for example, by prolonged continuous intravenous infusion.

The written instructions may also include a list of indications for which the dexmedetomidine hydrochloride composition is a suitable treatment.

### IX. Examples

### Example 1A

### Dosing of Dexmedetomidine Hydrochloride in a Middle-Aged to Elderly Cohort of Subjects

Dose escalation was performed by infusing 0.1 mcg/kg/hr to 0.6 mcg/kg/hr of dexmedetomidine hydrochloride intravenously over 30 min. Subjects in the initial cohorts were initially healthy, elderly (55-75 years of age) volunteers. Subjects in later cohort were elderly volunteers healthy except for having mild probable SDAT. Both sexes were included in all cohorts.

In the first cohort, dexmedetomidine hydrochloride was administered by IV infusion over a dose range from 0.1 mcg/kg/hr to 0.6 mcg/kg/hr. Each infusion rate was administered for 30 minutes. After 30 minutes, the dose was increased by an additional 0.1 mcg/kg/hr until the subject achieved a predetermined level of sedation as assessed by the Richmond Agitation-Sedation Scale (RASS) or a pre-specified reduction in BP and/or HR. The dosing rate was adjusted for subsequent cohorts based on the results of preceding cohorts. Continuous assessment was made of the level of arousal, BP and HR. Plasma samples were collected before dosing and then every 15 minutes for the determination of dexmedetomidine concentration. Statistics were descriptive and correlational in terms of pharmacodynamic and pharmacokinetic relationships.

Dexmedetomidine hydrochloride was found to achieve mild sedation (i.e. a RASS score of −1) in patients at a dexmedetomidine exposure level that did not produce clinically meaningful effects on blood pressure and/or heart rate. The effect was evident in 11/12 subjects on dexmedetomidine hydrochloride and occurred within 30 minutes of starting the dose which produced the desired effect. In contrast, a mild sedating effect was seen in only 1 out of 4 individuals on placebo. The mild sedative effects of dexmedetomidine hydrochloride persisted for 1.5 to 2 hours, a clinically relevant duration. Inter-individual variability of dexmedetomidine was limited by dose titration. The dexmedetomidine was well-tolerated.

Results are shown in Table 1 below

TABLE 1

| | | | | IV infusion rate at | | | | | |
|---|---|---|---|---|---|---|---|---|---|
| | | | | which desired | Cumulative | | | Plasma | Time |
| | | | | effect/stopping | dose (lowest | Plasma | | conc. at | needed to |
| Subject | | Age | Weight | criteria achieved, | to highest | conc. | | RS | resolve |
| Code | Gender | (Yrs) | (Kg) | (mcg/kg/hr) | (μg) | (pg/ml) | Endpoint | (pg/ml) | (mins) |
| 1201 | Female | 56 | 80.7 | 0.3 | 12 | 118.5 | RASS −1 | 41.8 | 117 |
| 1101 | | 73 | 51.5 | 0.4 | 18.4 | 114.42 | BP & HR > | 75.35 | 106 |
| | | | | | | | RASS −1 | | |
| 1106 | | 58 | 80.6 | 0.3 | 17.7 | 103.65 | RASS −2 | 44.2 | 135 |
| 9108 | | 57 | 70.1 | 0.3 | 19.7 | 182.73 | BP & HR > | 75.46 | 89 |
| | | | | | | | RASS −1 | | |
| 1202 | | 56 | 60.1 | 0.4 | 19.1 | 246.85 | BP > | 87.94 | 75 |
| | | | | | | | RASS −1 | | |
| 1205 | | 60 | 112.4 | 0.4 | 23.4 | 342.37 | RASS −1 > | 120.37 | 59 |
| | | | | | | | BP | | |
| 1207 | Male | 59 | 95.8 | 0.4 | 28.6 | 362.62 | RASS −1 | 72.02 | 115 |
| | | | | | | | & no BP/HR | | |
| 1105 | | 75 | 66.8 | 0.3 | 31.7 | 263.2 | BP & HR > | 52.86 | 148 |
| | | | | | | | RASS −1 | | |
| 1208 | | 56 | 81.9 | 0.4 | 33.4 | 270.22 | RASS −1 > | 91.92 | 89 |
| | | | | | | | HR | | |
| 9104 | | 58 | 88.7 | 0.4 | 41.7 | 406.27 | BP & HR > | 160.41 | >60* |
| | | | | | | | RASS −1 | | |
| 1103 | | 56 | 78.9 | 0.5 | 56.5 | 631.64 | BP & HR > | 169.24 | 102 |
| | | | | | | | RASS −1 | | |
| 1204 | | 66 | 72.7 | 0.4 | 25.4** | 320.51 | No RASS | 187.39 | NA** |
| | | | | | | | or BP/HR | | |

*time not captured by site;
**did not have an effect from one infusion;
RS: Resolution time.

Table 1 summarizes the effect of continuous IV infusion of dexmedetomidine hydrochloride on healthy elderly volunteers. Gender specific effects on responsiveness to dexmedetomidine was observed with females being more sensitive than males. The participants in cohort 1 were designated by subject codes 11XX and 91XX, and those in cohort 2 were designated by subject codes 12XX. Time needed to resolve refers to the time from the cessation of the infusion to the resolution of the pharmacodynamic effect of the drug on either arousal or blood pressure and heart rate.

The results of cohorts 1 and 2 established 0.3 mcg/kg/hr as the optimal starting dose. Plasma concentrations of the drug were correlated with both onset and offset of effect within a subject. There was inter-individual variability in response with approximately ⅓ being sensitive, ⅓ being intermediate, and ⅓ being insensitive. Gender affected drug responsiveness with males requiring twice the dose of dexmedetomidine hydrochloride compared to females (p<0.02, Sign test). Higher vagal tone (and therefore less sympathetic control) of blood pressure and heart rate may account for greater sensitivity to the cardiovascular effects of alpha-2 adrenergic receptor agonists and hence to the gender effect observed in this study.

These results confirm that arousable sedation with dexmedetomidine hydrochloride can be achieved without producing clinically meaningful effects on BP/HR.

### Example 1B

IV Dexmedetomidine Hydrochloride for Treating Acute Agitation in Patients with Senile Dementia of the Alzheimer's Type (SDAT)

We determined the optimal IV dose and safety profile of dexmedetomidine hydrochloride in the target population to achieve a RASS score of −1, how long the mild sedative effect persists after discontinuation of study drug administration, whether neurological effects like cognitive functioning, alertness/awareness, balance, and reaction time persist after the mild sedative effect has resolved, and whether any adverse effects on blood pressure, heart rate, or respiratory drive occurs before or coincident with the achievement of the aforementioned level of sedation.

This was a randomized, blinded, prospective, placebo controlled, parallel design, single centre, investigator-initiated study conducted to evaluate the efficacy and safety of dexmedetomidine hydrochloride in agitated subjects with senile dementia of Alzheimer's type (treatment allocation 5:2).

Clinical evaluation was performed as follows. Patients were screened up to 4 weeks prior to randomization. After obtaining the written informed consent from subjects, subjects were allowed to participate in the study related screening activities. Screening activities included clinical laboratory tests, physical examination, ECG, etc. After completion of all screening related activities, the eligible subjects [male and female subjects 55 to 75 years of age; known cases of mild probable Alzheimer's disease (SDAT) in accordance with NINCDS/ADRDA criteria for diagnosis of Alzheimer's disease with a Mini-Mental State Score of between 20 to 24; RASS score of +1 or +2 on the Day −1 and prior to drug administration on Day 1] were randomized in the study (treatment allocation 5:2). Subjects were housed in the clinic from Day −1 (preferably in the afternoon or evening) to end of study. Additional housing was allowed performed based on the investigator's discretion.

Day 1: Day of randomization and end of study

Day 3: Safety follow up

Study drug IV infusion was to be stopped at any time if any of the following occurred:
There was more than 20 mm Hg decrease in systolic or diastolic blood pressure.
There was an isolated drop in Systolic BP<100 mm of Hg.
There was an isolated drop Diastolic BP<60 mm of Hg.
There was a decrease in heart rate of 20 beats per minute or a drop below 50 beats per minute.
Attainment of a RASS of −1 at the end of each 30 minutes infusion period.

If one of the above stopping criteria was met, the site stopped the infusion and continued to monitor the participant's vital signs every 15 minutes until the participant reached their baseline parameters or, in the judgment of the principal investigator, the participant had reached a stable and acceptable level of blood pressure and heart rate. Return to baseline parameters was defined as BP falling within 15 mm of Hg of baseline reading prior to drug administration or HR falling within 10 beats per minute of baseline reading prior to drug administration.

RASS was assessed every 15 minutes until the participant reached a RASS of 0 or, in exceptional circumstances, a stable and acceptable level of arousal was met in the judgment of principle investigator. All raters were trained in the use of the RASS scale.

Clinical assessments of cognitive functioning, alertness/awareness, balance and reaction time were performed prior to the IV infusion, at the end of infusion until the participant had reached their baseline parameters or, in the judgment of the principal investigator, the participant had reached a stable and acceptable level of arousal.

Blood samples (each of 2 mL) were collected at pre-dose (within 5 minutes of study drug administration), every 15 minutes during the IV infusion, and at the time the subject was judged to have fully recovered from the effects of the infused drug. These samples were assayed for the concentration of the drug in those participants randomized to test product.

Schedule of study procedure is described below in Table 2

TABLE 2
Schedule of Study Procedures

| | Screening | | | | | |
|---|---|---|---|---|---|---|
| | Period | | | | | Safety |
| | Up to 4 weeks | | Randomization | | | Follow |
| | prior to | | Pre-dose | Post-dose | End of | up |
| Day | randomization | Day −1^{a} | (Day 1) | (Day 1) | Study^{b} | Day 3 |
| Informed Consent | X | | | | | |
| Demographic data | X | | | | | |
| Medical History | X | | | | | |
| Physical examination | X | X | | | X | |
| Vital sign recording | X | X | within | every 15 | X | |
| | | | 30 | minutes | | |
| | | | minutes | throughout the | | |
| | | | prior to | infusion and | | |
| | | | dosing | until the | | |
| | | | | participant has | | |
| | | | | reached their | | |
| | | | | baseline | | |
| | | | | parameters are | | |
| | | | | achieved | | |
| Inclusion/Exclusion | X | X | X | | | |
| Criteria Review | | | | | | |
| Urine Drug screen | X | X | | | | |
| Clinical Laboratory | X | X | | | X | |
| Evaluation | | | | | | |
| 12 lead ECG^{c} | X | X | within | | Within | |
| | | | 30 | | 10 | |
| | | | minutes | | minutes | |
| | | | prior to | | of end | |
| | | | dosing | | of | |
| | | | | | infusion | |
| Adverse Event | X | X | X | X | X | X |
| Assessment | (after signing | | | | | |
| | ICF) | | | | | |
| Drug administration | | | | Initial dose: | | |
| | | | | 0.1 pg/kg/hr. | | |
| | | | | The dose can | | |
| | | | | be increased | | |
| | | | | by up to 0.1 | | |
| | | | | pg/kg/hr if the | | |
| | | | | desired | | |
| | | | | reduced in | | |
| | | | | agitation has | | |
| | | | | not been | | |
| | | | | achieved by | | |
| | | | | the end of 30 | | |
| | | | | minutes (+2 | | |
| | | | | minutes) of | | |
| | | | | infusion of the | | |
| | | | | prior dose | | |
| Richmond | X | X | X | every 15 | | |
| Agitation-Sedation | | | | minutes until | | |
| Scale (RASS) | | | | the participant | | |
| Evaluation | | | | has reached a | | |
| | | | | RASS of 0 or | | |
| | | | | in exceptional | | |
| | | | | circumstances | | |
| | | | | a stable and | | |
| | | | | acceptable | | |
| | | | | level of | | |
| | | | | arousal in the | | |
| | | | | judgment of | | |
| | | | | the principle | | |
| | | | | investigator | | |
| Clinical Assessment | | | X | X | | |
| of cognitive | | | | | | |
| functioning, | | | | | | |
| alertness/awareness, | | | | | | |
| balance and reaction | | | | | | |
| time | | | | | | |

^{a}If interval between screening visit and Day −1 was seven days or less, then laboratory investigations on Day −1 were waived.
^{b}End of study activity were performed at least 2 hours after resolution of any reduction in tire level of drowsiness and/or meeting stopping criteria related to HR and BP.
^{c}On each time point, site team performed triplicate ECG to ensure the accurate reading. QTc interval using the Bazett's formula was recorded
Note
Screening and randomization visit was performed on same day.

### Treatments

There were two treatment arms in the study as described below in Table 3.

TABLE 3
Study treatment

| Treatment Arms | Intervention |
|---|---|
| Test Product | Dexmedetomidine hydrochloride injection |
| Test Placebo | Normal Saline |

### Treatments Administered

The initial dose of dexmedetomidine hydrochloride was 0.1 mcg/kg/hr, with no loading dose, with the desired endpoint being the attainment of a RASS score of −1. Once the participant was drowsy but able to respond to verbal stimulation, the infusion was stopped, and time was recorded. The dose was increased by up to 0.1 mcg/kg/hr if the desired level of sedation was not achieved by the end of 30 minutes of IV infusion of the prior dose. The infusion was continued for up to 150 minutes to the maximum dose. In no instance was the infusion rate allowed to exceed 0.5 mcg/kg/hr and the maximum total dose administered was capped to 0.75 mcg/kg/hr.

### Statistical Methods Planned in the Protocol

Statistical analysis and the associated tables, listings and figures were performed using SAS® (SAS Institute Inc., Cary, USA) version 9.4. Continuous variables, such as age, were summarized with descriptive statistic (n, mean, SD, minimum, maximum) and categorical variable, such as sex, with n (%). Efficacy parameters were represented by descriptive statistics (n, mean, median, SD, minimum, maximum).

Efficacy and safety analysis were performed on all the randomized subjects.

### Results:

### Demographic and Other Baseline Characteristics

Asian subjects with mild probable Alzheimer's disease (SDAT) in accordance with NINCDS/ADRDA criteria were randomized in the study. Mini-Mental State Score of randomized subjects ranged from 20-24.

The mean age, mean height, mean weight and mean BMI were 65.3 years, 151.4 cm, 51.13 kg and 22.060 kg/m² respectively for the subjects randomized to dexmedetomidine hydrochloride treatment group. The mean age, mean weight and mean BMI were 68.3 years, 157.5 cm, 63.25 kg and 26.053 kg/m² respectively for the subjects randomized to placebo treatment group. Overall, demographic characteristic of randomized subjects was comparable between two treatment groups.

Summary of subject demographic characteristics is presented in Table 4.

TABLE 4
depicts the summary of Subject Demographic Characteristics

| Demographic | | Test | | Placebo | | Overall | |
|---|---|---|---|---|---|---|---|
| Characteristics | Statistics | (N = 10) | | (N = 4) | | (N = 14) | |
| Age (Years) | N | 10 | | 4 | | 14 | |
| | Mean ± SD | 65.3 ± 7.36 | | 68.3 ± 9.07 | | 66.1 ± 7.64 | |
| | Median | 65.5 | | 71.5 | | 67.0 | |
| | Min, Max | 55, 75 | | 55, 75 | | 55, 75 | |
| Sex [n (%)] | Male | 4 | (40.00) | 3 | (75.00) | 7 | (50.00) |
| | Female | 6 | (60.00) | 1 | (25.00) | 7 | (50.00) |
| Race [n (%)] | Asian | 10 | (100.00) | 4 | (100.00) | 14 | (100.00) |
| | American Indian | 0 | (0.00) | 0 | (0.00) | 0 | (0.00) |
| | or Alaska Native | | | | | | |
| | White | 0 | (0.00) | 0 | (0.00) | 0 | (0.00) |
| | Native Hawaiian | 0 | (0.00) | 0 | (0.00) | 0 | (0.00) |
| | or Other Pacific | | | | | | |
| | Islander | | | | | | |
| | Black or African | 0 | (0.00) | 0 | (0.00) | 0 | (0.00) |
| | American | | | | | | |
| | Other | 0 | (0.00) | 0 | (0.00) | 0 | (0.00) |

| | | | | |
|---|---|---|---|---|
| Height (cm) | N | 10 | 4 | 14 |
| | Mean ± SD | 151.4 ± 9.18 | 157.5 ± 10.41 | 153.1 ± 9.57 |
| | Median | 150.5 | 157.5 | 152.0 |
| | Min, Max | 139, 166 | 146, 169 | 139, 169 |
| Weight (kg) | N | 10 | 4 | 14 |
| | Mean ± SD | 51.13 ± 11.392 | 63.25 ± 7.805 | 54.59 ± 11.670 |
| | Median | 46.70 | 62.00 | 54.95 |
| | Min, Max | 40.0, 72.0 | 56.0, 73.0 | 40.0, 73.0 |
| BMI (kg/m²) | N | 10 | 4 | 14 |
| | Mean ± SD | 22.060 ± 2.6849 | 26.053 ± 6.6141 | 23.201 ± 4.3115 |
| | Median | 21.030 | 24.825 | 21.095 |
| | Min, Max | 18.92, 27.78 | 20.31, 34.25 | 18.92, 34.25 |

Abbreviations: BMI = body mass index; N = number of subjects in respective treatment; n = number of subjects in specified category.
Note:
Percentages are based on the number of subjects in the specified treatment.

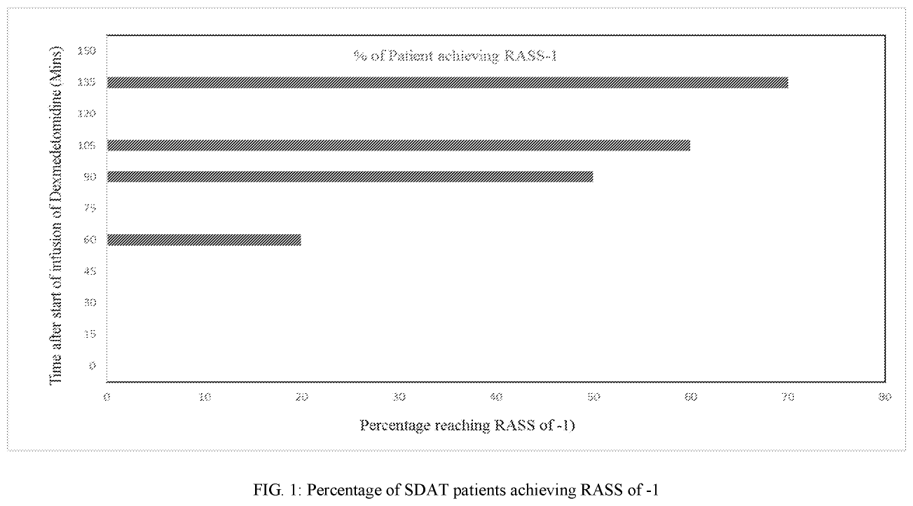

Primary endpoint—RASS score −1 (Dose at which drowsiness can be achieved which can be temporarily reversed by verbal stimulation). Results demonstrated that 7 (Patient No. 101, 105, 106, 109, 110, 111, 113) out of 10 subjects achieved a RASS score of −1 with dexmedetomidine hydrochloride IV infusion (see Table 5A, 5B and FIG. 1), while only 1 (Patient No. 107) out of 4 subjects achieved a RASS score of −1 with placebo IV infusion. The IV infusion rate of dexmedetomidine hydrochloride ranged from about 0.2 to about 0.5 mcg/kg/hr with a mean infusion rate 0.34 mcg/kg/hr.

Summary of optimal dose (mcg/kg/hr) at which a RASS score of −1 was achieved is presented in Table 5A.

TABLE 5A

| | Test |
|---|---|
| Statistic | (N = 10) |
| N | 7 |
| Mean ± Sd | 0.34 ± 0.098 |
| Median | 0.30 |
| Mode | 0.30 |
| Min, Max | 0.2, 0.5 |

Abbreviations: N = number of subjects in respective treatment group; n = number of subjects in specified category; RASS = Richmond agitation sedation scale;
Note:
Subject was considerered as drowsy when their RASS reached −1.

TABLE 5B

| | RASS score | | | | | | | | | | |
|---|---|---|---|---|---|---|---|---|---|---|---|
| | 00:00 | 00:15 | 00:30 | 00:45 | 01:00 | 01:15 | 01:30 | 01:45 | 02:00 | 02:15 | 02:30 |
| Subject No. | Hr | Hr | Hr | Hr | Hr | Hr | Hr | Hr | Hr | Hr | Hr |
| 101 (Treatment) | 1 | 1 | 1 | 1 | −1 | | | | | | |
| 102 (Treatment) | 1 | 0 | 0 | 0 | 1 | 1 | 0 | | | | |
| 103 (Placebo) | 1 | 1 | 0 | 0 | 0 | 0 | 0 | 0 | 0 | 0 | 0 |
| 104 (Treatment) | 2 | 2 | 1 | 1 | 1 | 1 | 1 | 1 | 1 | 1 | 1 |
| 105 (Treatment) | 2 | 2 | 1 | 1 | 1 | 0 | −1 | | | | |
| 106 (Treatment) | 2 | 2 | 1 | 1 | 1 | 0 | −1 | | | | |
| 107 (Placebo) | 1 | 0 | −1 | | | | | | | | |
| 108 (Placebo) | 1 | 1 | 1 | 1 | 1 | 0 | 0 | 1 | 1 | 1 | 1 |
| 109 (Treatment) | 1 | 1 | 1 | 0 | −1 | | | | | | |
| 110 (Treatment) | 1 | 1 | 1 | 1 | 0 | 0 | 0 | −1 | | | |
| 111 (Treatment) | 2 | 2 | 1 | 1 | 1 | 1 | 1 | 0 | 0 | −1 | |
| 112 (Treatment) | 2 | 1 | 1 | 1 | 1 | 1 | 1 | 1 | 1 | 1 | 1 |
| 113 (Treatment) | 1 | 1 | 0 | 0 | 0 | 0 | −1 | | | | |
| 114 (Placebo) | 1 | 1 | 1 | 0 | 0 | 1 | 1 | 1 | 1 | 1 | 1 |

Secondary endpoint: Time period for which a calming effect persisted after discontinuation of the study drug administration. Subjects became alert and remained calm after around 30 minutes of dexmedetomidine infusion discontinuation of study drug (see Table 6).

TABLE 6

| | Test | Placebo |
|---|---|---|
| Statistic | (N = 10) | (N = 4) |
| N | 7 | 1 |
| Mean ± SD | 28.6 ± 15.74 | NE |
| Median | 30 | NE |
| Min, Max | 5, 45 | NE |

Abbreviations: N = number of subjects in respective treatment group; n = number of subjects in specified category; NE = not estimable; RASS = Richmond agitation sedation scale.

Time period at which clinical assessment of cognitive functioning, alertness/awareness, balance, and reaction time persist after drowsiness has resolved was assessed. Cognitive functioning, alertness/awareness, balance, and reaction time reached to their baseline level within around 45 minutes after drowsiness was resolved/recovered (see Table 7).

TABLE 7

| | Test | Placebo |
|---|---|---|
| Statistic | (N = 10) | (N = 4) |
| N | 7 | 1 |
| Mean ± SD | 42.9 ± 27.36 | NE |
| Median | 45 | NE |
| Min, Max | 10,90 | NE |

Abbreviations: N = number of subjects in respective treatment group; n = number of subjects in specified category; NE = not estimable.
Note:
Time of post-dose assessment of cognitive function - RASS actual time when drowsiness is achieved.

The time at which the participant was judged suitable for discharge after infusion was stopped was a return to their baseline level of alertness and awareness with no impairment in balance, gait, and reaction time as determined by the principal investigator or designee.

All subjects were discharged once the RASS score achieved 0 or once vital signs reached to their baseline value as per the investigator's judgment, and no additional housing was required as per the investigator's discretion.

### Safety Evaluations

### Extend of Exposure:

Total dose of dexmedetomidine hydrochloride ranged from 0.15-0.75 mcg/kg. Mean infusion rate of dexmedetomidine hydrochloride ranged from 0.2-0.5 mcg/kg/hr.

### Adverse Events and Other Important Safety Findings:

No adverse event was reported during the study.

As per the safety stoppage rule, only one subject's systolic BP reduced by more than 20 mm of Hg, which returned to baseline levels within 10 minutes after infusion was stopped (See Table 8).

TABLE 8

| Subject No | Treatment | Subject stopping Criteria |
|---|---|---|
| 102 | Test | SBP >20 mm of Hg decrease |

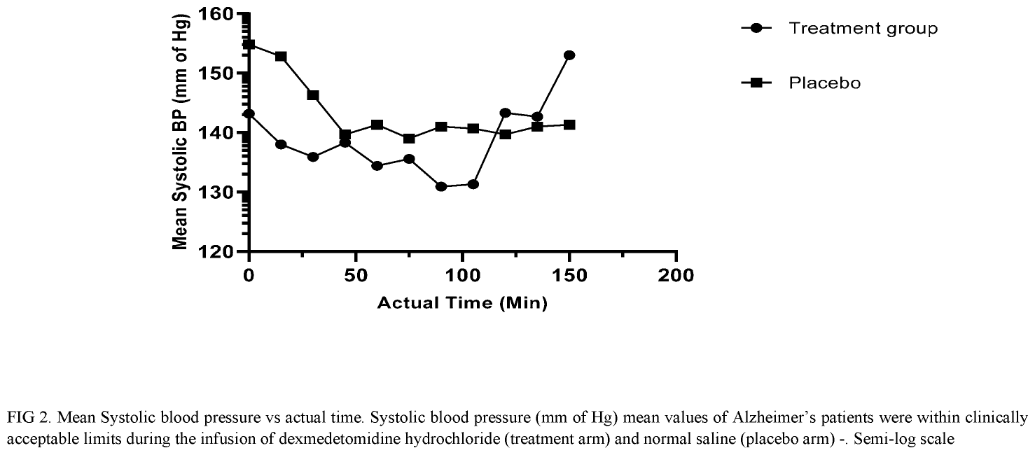

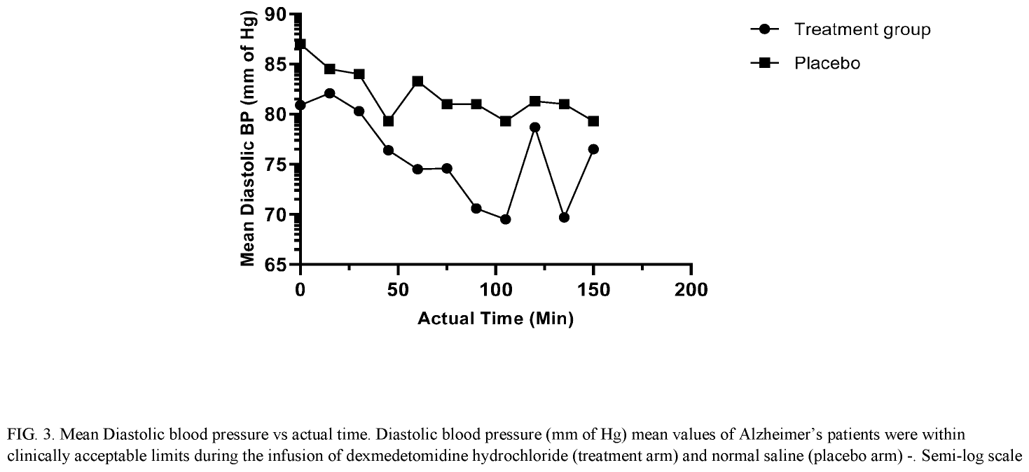

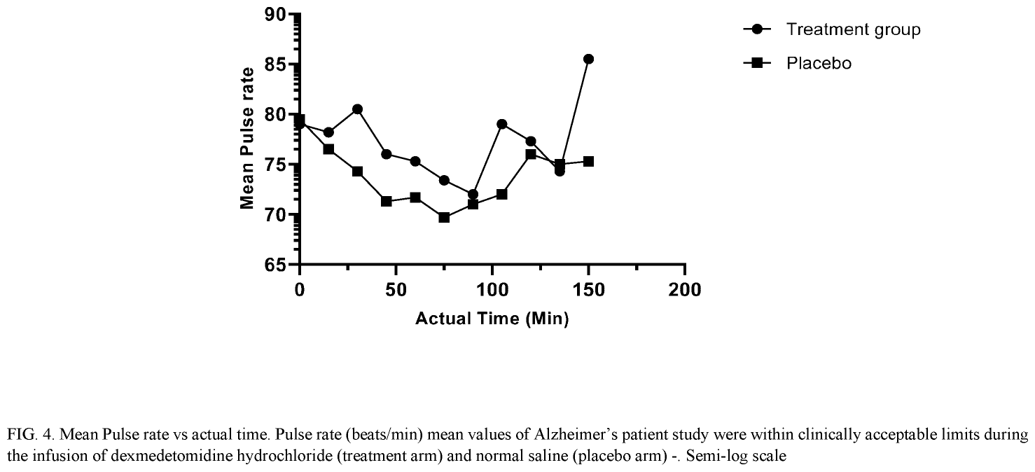

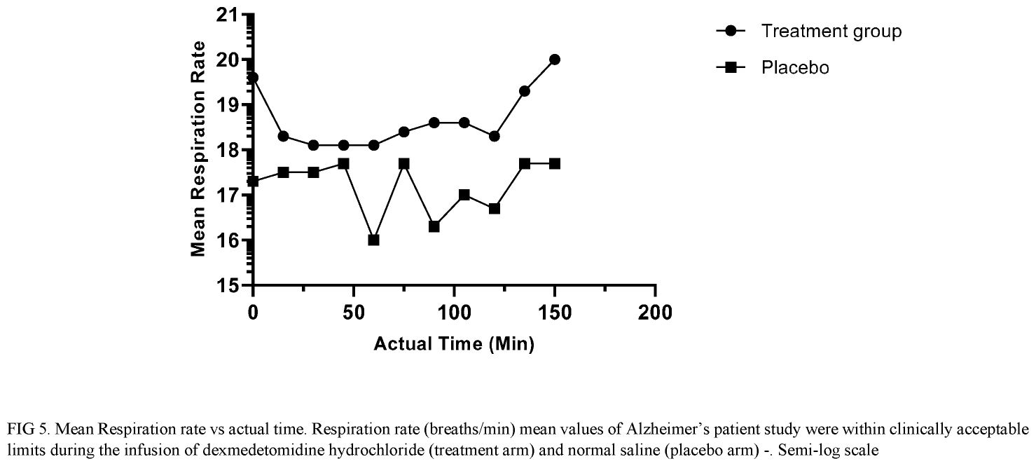

Summary of vital signs of Alzheimer's patients (SDAT) is presented in FIGS. 2, 3, 4 and 5 and also tabulated in tables in 9A, 9B, 9C, 9D and 9E. All the vital signs values were within clinically acceptable limits.

Systolic blood pressure (mm of Hg) mean values of Alzheimer's patient study were within clinically acceptable limits during the infusion of dexmedetomidine hydrochloride and normal saline (see Table 9A).

TABLE 9A

| | | | systolic blood pressure (mm of Hg) | | | | |
|---|---|---|---|---|---|---|---|
| Treatment | | | 00:00 | 00:15 | 00:30 | 00:45 | 01:00 |
| group | Statistics | Screening | Hr | Hr | Hr | Hr | Hr |
| Dex | n | 10 | 10 | 10 | 10 | 10 | 10 |
| | Mean ± SD | 126.4 ± 10.42 | 143.2 ± 13.60 | 138.0 ± 10.35 | 135.9 ± 10.05 | 138.3 ± 14.29 | 134.4 ± 15.76 |
| | Median | 125.0 | 140.0 | 134.5 | 133.5 | 137.0 | 133.0 |
| | Min, Max | 110, 145 | 125, 169 | 122, 155 | 120, 155 | 118, 160 | 110, 160 |
| Placebo | n | 4 | 4 | 4 | 4 | 3 | 3 |
| | Mean ± SD | 149.3 ± 17.00 | 154.8 ± 12.74 | 152.8 ± 10.31 | 146.3 ± 11.09 | 139.7 ± 9.07 | 141.3 ± 9.81 |
| | Median | 151.0 | 159.5 | 155.5 | 147.5 | 141.0 | 147.0 |
| | Min, Max | 130, 165 | 136, 164 | 138, 162 | 132, 158 | 130, 148 | 130, 147 |
| | | systolic blood pressure (mm of Hg) | | | | | |
| Treatment | | 01:15 | 01:30 | 01:45 | 02:00 | 02:15 | 02:30 |
| group | Statistics | Hr | Hr | Hr | Hr | Hr | Hr |
| Dex | n | 8 | 8 | 4 | 3 | 3 | 2 |
| | Mean ± SD | 135.6 ± 13.94 | 130.9 ± 13.44 | 131.3 ± 23.43 | 143.3 ± 17.01 | 142.7 ± 20.98 | 153.0 ± 1.41 |
| | Median | 141.0 | 129.5 | 131.0 | 150.0 | 150.0 | 153.0 |
| | Min, Max | 110, 152 | 112, 156 | 105, 158 | 124, 156 | 119, 159 | 152, 154 |
| Placebo | n | 3 | 3 | 3 | 3 | 3 | 3 |
| | Mean ± SD | 139.0 ± 4.36 | 141.0 ± 4.36 | 140.7 ± 9.24 | 139.7 ± 4.04 | 141.0 ± 1.73 | 141.3 ± 7.02 |
| | Median | 141.0 | 143.0 | 146 | 142.0 | 140.0 | 142.0 |
| | Min, Max | 134, 142 | 136, 144 | 130, 146 | 135, 142 | 140, 143 | 134, 148 |

Diastolic blood pressure (mm of Hg) mean values of Alzheimer's patient study were within clinically acceptable limits during the infusion of dexmedetomidine hydrochloride and normal saline (see Table 9B)

TABLE 9B

| | | | Diastolic blood pressure (mm of Hg) | | | | |
|---|---|---|---|---|---|---|---|
| Treatment | | | 00:00 | 00:15 | 00:30 | 00:45 | 01:00 |
| group | Statisties | Screening | Hr | Hr | Hr | Hr | Hr |
| Dex | N | 10 | 10 | 10 | 10 | 10 | 10 |
| | Mean ± SD | 81.8 ± 6.76 | 80.9 ± 9.87 | 82.1 ± 8.46 | 80.3 ± 7.99 | 76.4 ± 9.42 | 74.5 ± 8.97 |
| | Median | 80.0 | 83.5 | 84.0 | 82.5 | 79.0 | 73.5 |
| | Min, Max | 68, 90 | 64, 90 | 70, 92 | 66, 89 | 62, 86 | 63, 90 |
| Placebo | N | 4 | 4 | 4 | 4 | 3 | 3 |
| | Mean ± SD | 80.0 ± 14.14 | 87.0 ± 3.46 | 84.5 ± 7.85 | 84.0 ± 5.60 | 79.3 ± 8.02 | 83.3 ± 10.79 |
| | Median | 85.0 | 87.0 | 87.5 | 85.0 | 80.0 | 88.0 |
| | Min, Max | 60, 90 | 84, 90 | 73, 90 | 77, 89 | 71, 87 | 71, 91 |
| | | Diastolic blood pressure (mm of Hg) | | | | | |
| Treatment | | 01:15 | 01:30 | 01:45 | 02:00 | 02:15 | 02:30 |
| group | Statisties | Hr | Hr | Hr | Hr | Hr | Hr |
| Dex | N | 8 | 8 | 4 | 3 | 3 | 2 |
| | Mean ± SD | 74.6 ± 10.17 | 70.6 ± 8.14 | 69.5 ± 11.82 | 78.7 ± 11.02 | 69.7 ± 13.28 | 76.5 ± 9.19 |
| | Median | 79.5 | 70.0 | 66.0 | 84.0 | 62.0 | 76.5 |
| | Min, Max | 61, 84 | 62, 84 | 60, 86 | 66, 86 | 62, 85 | 70, 83 |
| Placebo | N | 3 | 3 | 3 | 3 | 3 | 3 |
| | Mean ± SD | 81.0 ± 7.55 | 81.0 ± 8.89 | 79.3 ± 11.59 | 81.3 ± 4.62 | 81.0 ± 2.65 | 79.3 ± 1.15 |
| | Median | 82.0 | 84.0 | 85.0 | 84.0 | 82.0 | 80.0 |
| | Min, Max | 73, 88 | 71, 88 | 66, 87 | 76, 84 | 78, 83 | 78, 80 |

Pulse rate (beats/min) mean values of Alzheimer's patient study were within clinically acceptable limits during the infusion of dexmedetomidine hydrochloride and normal saline (see Table 9C)

TABLE 9C

| | | | Pulse rate (beats/min) | | | | |
|---|---|---|---|---|---|---|---|
| | | | 00:00 | 00:15 | 00:30 | 00:45 | 01:00 |
| Subject | Statistics | Screening | Hr | Hr | Hr | Hr | Hr |
| Dex | N | 10 | 10 | 10 | 10 | 10 | 10 |
| | Mean ± SD | 80.2 ± 6.00 | 79.0 ± 9.02 | 78.2 ± 9.85 | 80.5 ± 10.43 | 76.0 ± 9.48 | 75.3 ± 10.08 |
| | Median | 80.0 | 78.0 | 80.0 | 76.5 | 77.0 | 74.0 |
| | Min, Max | 72, 92 | 64, 91 | 57, 89 | 64, 94 | 58, 90 | 60, 89 |
| Placebo | N | 4 | 4 | 4 | 4 | 3 | 3 |
| | Mean ± SD | 75.8 ± 12.28 | 79.5 ± 9.68 | 76.5 ± 10.79 | 74.3 ± 8.42 | 71.3 ± 8.08 | 71.7 ± 8.50 |
| | Median | 74.0 | 78.0 | 75.0 | 74.0 | 70.0 | 72.0 |
| | Min, Max | 65, 90 | 71, 91 | 67, 89 | 65, 84 | 64, 80 | 63, 80 |
| | | Pulse rate (beats/min) | | | | | |
| | | 01:15 | 01:30 | 01:45 | 02:00 | 02:15 | 02:30 |
| Subject | Statistics | Hr | Hr | Hr | Hr | Hr | Hr |
| Dex | N | 8 | 8 | 5 | 3 | 3 | 2 |
| | Mean ± SD | 73.4 ± 11.56 | 72.0 ± 14.66 | 79.0 ± 14.23 | 77.3 ± 20.82 | 74.3 ± 21.13 | 85.5 ± 14.85 |
| | Median | 71.5 | 71.0 | 79.0 | 84.0 | 77.0 | 85.5 |
| | Min, Max | 58, 94 | 52, 94 | 61, 100 | 54, 94 | 52, 94 | 75, 96 |
| Placebo | N | 3 | 3 | 3 | 3 | 3 | 3 |
| | Mean ± SD | 69.7 ± 10.97 | 71.0 ± 8.89 | 72.0 ± 10.54 | 76.0 ± 7.21 | 75.0 ± 8.00 | 75.3 ± 7.02 |
| | Median | 66.0 | 68.0 | 71.0 | 74.0 | 75.0 | 76.0 |
| | Min, Max | 61, 82 | 64, 81 | 62. 83 | 70, 84 | 67, 83 | 68, 82 |

Respiration rate (breaths/min)) values of Alzheimer's patient study were within clinically acceptable limits during the infusion of dexmedetomidine hydrochloride and normal saline (see Table 9D)

TABLE 9D

| | | | Respiration rate (breaths/min)) | | | | |
|---|---|---|---|---|---|---|---|
| Subject | | | 00:00 | 00:15 | 00:30 | 00:45 | 01:00 |
| Type | Statistics | Screening | Hr | Hr | Hr | Hr | Hr |
| Dex | N | 10 | 10 | 10 | 10 | 10 | 10 |
| | Mean ± SD | 18.2 ± 1.69 | 19.6 ± 2.50 | 18.3 ± 3.20 | 18.1 ± 2.73 | 18.1 ± 1.91 | 18.1 ± 1.73 |
| | Median | 19.0 | 19.5 | 17.0 | 18.0 | 18.0 | 18.0 |
| | Min, Max | 15, 20 | 17, 26 | 15, 26 | 13, 24 | 15, 20 | 15, 20 |
| Placebo | N | 4 | 4 | 4 | 4 | 3 | 3 |
| | Mean ± SD | 17.8 ± 0.96 | 17.3 ± 3.59 | 17.5 ± 1.73 | 17.5 ± 2.52 | 17.7 ± 1.53 | 16.0 ± 2.00 |
| | Median | 17.5 | 18.5 | 17.0 | 18.0 | 18.0 | 16.0 |
| | Min, Max | 17, 19 | 12, 20 | 16, 20 | 14, 20 | 16, 19 | 14, 18 |
| | | Respiration rate (breaths/min)) | | | | | |
| Subject | | 01:15 | 01:30 | 01:45 | 02:00 | 02:15 | 02:30 |
| Type | Statistics | Hr | Hr | Hr | Hr | Hr | Hr |
| Dex | N | 8 | 8 | 5 | 3 | 3 | 2 |
| | Mean ± SD | 18.4 ± 2.20 | 18.6 ± 3.38 | 18.6 ± 2.51 | 18.3 ± 1.53 | 19.3 ± 1.15 | 20.0 ± 0.00 |
| | Median | 19.5 | 19.0 | 18.0 | 18.0 | 20.0 | 20.0 |
| | Min, Max | 15, 20 | 15, 25 | 17, 23 | 17, 20 | 18, 20 | 20, 20 |
| Placebo | N | 3 | 3 | 3 | 3 | 3 | 3 |
| | Mean ± SD | 17.7 ± 1.53 | 16.3 ± 2.89 | 17.0 ± 2.65 | 16.7 ± 1.53 | 17.7 ± 2.52 | 17.7 ± 2.52 |
| | Median | 18.0 | 18.0 | 18.0 | 17.0 | 18.0 | 18.0 |
| | Min, Max | 16, 19 | 13, 18 | 14, 19 | 15, 18 | 15, 20 | 15, 20 |

TABLE 9E
depicts QTc interval values of Alzheimer's patient study

| | | | Test | Placebo |
|---|---|---|---|---|
| Test Name | Visit | Statistic | (N = 10) | (N = 4) |
| ECG | Screening | N | 10 | 4 |
| | | Mean ± SD | 442.0 ± 24.76 | 433.2 ± 13.81 |
| | | Median | 440.7 | 433.7 |
| | | Min, Max | 398, 477 | 416, 449 |
| Pre-ECG | Before | N | 10 | 4 |
| | Dosing | Mean ± SD | 447.2 ± 35.51 | 440.9 ± 31.85 |
| | | Median | 444.3 | 443.3 |
| | | Min, Max | 398, 524 | 400, 477 |
| Post-ECG | After | N | 10 | 4 |
| | stopping | Mean ± SD | 462.9 ± 73.04 | 442.5 ± 26.58 |
| | of Dose | Median | 441.0 | 450.5 |
| | | Min, Max | 370, 592 | 405, 464 |

Abbreviations: N = number of subjects in the specified treatment; n = number of subjects in specified category.

Plasma concentrations were measured for all subjects at intervals of 15 minutes until 180 minutes total duration was reached, and the results tabulated in Table 10. The maximum dexmedetomidine concentrations in Alzheimer's patients ranged from 102 pg/ml to 380 pg/ml. Time to reach Cmax in ranged from 60 minutes to 150 minutes. The mean infusion rate is 0.34 mcg/kg/hr with the maximum rate ranging from 0.2 to 0.5 mcg/kg/hr.

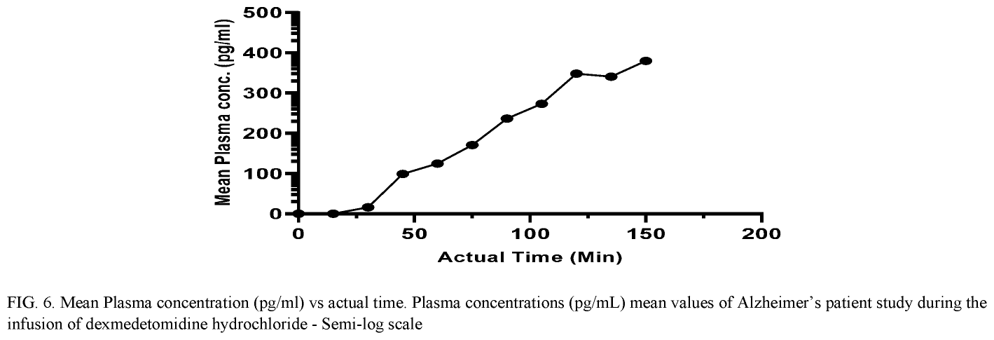

Table 10 and FIG. 6: depicts the plasma concentrations (pg/mL) of Alzheimer's (SDAT) patients

| | Plasma concentrations (pg/mL) | | | | | | | | | | |
|---|---|---|---|---|---|---|---|---|---|---|---|
| | 00:00 | 00:15 | 00:30 | 00:45 | 01:00 | 01:15 | 01:30 | 01:45 | 02:00 | 02:15 | 02:30 |
| Subject No. | Hr | Hr | Hr | Hr | Hr | Hr | Hr | Hr | Hr | Hr | Hr |
| 101 (Treatment) | 0.00 | 0.00 | 27.19 | 94.76 | 101.97 | x | x | x | x | x | x |
| 102 (Treatment) | 0.00 | 0.00 | 13.86 | 47.23 | 79.54 | 118.61 | 183.68 | x | x | x | x |
| 103 (Placebo) | 0.00 | 0.00 | 0.00 | 0.00 | 0.00 | 0.00 | 0.00 | 0.00 | 0.00 | 0.00 | 0.00 |
| 104 (Treatment) | 0.00 | 0.00 | 0.00 | 62.72 | 85.22 | 159.58 | 210.69 | 249.92 | 318.05 | 347.97 | 379.92 |
| 105 (Treatment) | 0.00 | 0.00 | 0.00 | 158.40 | 176.64 | 222.59 | 239.54 | x | x | x | x |
| 106 (Treatment) | 0.00 | 0.00 | 37.05 | 102.76 | 126.21 | 192.31 | 176.26 | x | x | x | x |
| 107 (Placebo) | 0.00 | 0.00 | 0.00 | 0.00 | 0.00 | 0.00 | 0.00 | 0.00 | 0.00 | 0.00 | 0.00 |
| 108 (Placebo) | 0.00 | 0.00 | 0.00 | 0.00 | 0.00 | 0.00 | 0.00 | 0.00 | 0.00 | 0.00 | 0.00 |
| 109 (Treatment) | 0.00 | 0.00 | 12.72 | 138.71 | 121.54 | x | x | x | x | x | x |
| 110 (Treatment) | 0.00 | 0.00 | 23.32 | 120.73 | 178.86 | 154.35 | 316.27 | 258.44 | x | x | x |
| 111 (Treatment) | 0.00 | 0.00 | 13.47 | 67.73 | 129.62 | 177.23 | 291.68 | 311.46 | 378.16 | 333.56 | x |
| 112 (Treatment) | NA | NA | NA | NA | NA | NA | NA | NA | NA | NA | NA |
| 113 (Treatment) | NA | NA | NA | NA | NA | NA | NA | NA | NA | NA | NA |
| 114 (Placebo) | 0.00 | 0.00 | 0.00 | 0.00 | 0.00 | 0.00 | 0.00 | 0.00 | 0.00 | 0.00 | 0.00 |

NA—Not analyzed

### Example 2

IV Dexmedetomidine Hydrochloride in Subjects Suffering from Schizophrenia

The Primary Objective was to determine the optimal intravenous (IV) dose of dexmedetomidine hydrochloride in the target population in terms of efficacy and safety to achieve arousable sedation (RASS of −1) which can be reversed by verbal stimulation. When this goal was achieved in each participant, the IV infusion of dexmedetomidine hydrochloride ceased.

In addition, we assessed the following Secondary Objectives:
a. Determine how rapidly the drug can be administered up to the total dose needed to achieve RASS −1.
b. Determine how long the calming effect persists after discontinuation of study drug administration.
c. Determine whether any adverse effects on blood pressure, heart rate, or respiratory drive occurs before or coincident with the achievement of Primary Objective. Stopping rules for blood pressure and heart rate, indicating a clinically significant event, are:
   1. drop in systolic BP<90 mm of Hg.
   2. drop in diastolic BP<60 mm of Hg
   3. drop below 50 beats per minute

Participants were provided written informed consent before any study related procedures were performed. All participants were screened for inclusion and exclusion criteria. The participants were admitted to the site at screening (Day −1), the day before the infusion. Baseline assessments were performed on Day −1, as well as on the day of infusion (Day 1). The participants were on Day 1 prepared for the infusion, infused for up to 3 hours and monitored for resolution of sedation and any decreases in blood pressure or heart rate which met stopping criteria. The participants were not discharged from the research unit until three hours after resolution of any reduction in the level of arousal (e.g., RASS −1) and/or resolution of any decrease in blood pressure or heart rate meeting stopping criteria. The Principal Investigator had discretion to keep the participant overnight at the site the evening of Day 1 for extended monitoring and then discharge home the participant on Day 2 if the Principal Investigator or designee determined that the participant has returned to their baseline state.

The study population included 14 participants, 10 active and 4 placebo. Patients 5, 7, 8 and 9 received placebo. Patients 1, 2, 3, 4, 11, 12, 14, 16, 17, 18 were infused with intravenous dexmedetomidine hydrochloride, starting at a rate of 0.2 mcg/kg/hr, and rising by 0.1 mcg/kg/hr every 30 minutes until stopping criteria were reached up or to a maximum duration of 3 hours. Participants randomized to placebo received a matching intravenous infusion of placebo solution.

TABLE 11
Study Treatments

| Treatment | Formulation | Frequency |
|---|---|---|
| Dexmedetomidine | Precedex ® | Continuous infusion, increment every |
| hydrochloride | | 30 minutes |
| Placebo | Normal Saline | Continuous infusion |

Once the participant was drowsy (RASS −1), the infusion was stopped. The maximum total dose administered was 1.6 mcg/kg/hr, when either the desired level of sedation was achieved or the maximum allowable decrease in either systolic or diastolic blood pressure or heart rate occurred.

The participants were continuously monitored during the study by the site personnel, including monitoring blood pressure and heart rate. Intermittent electrocardiograms were taken from the start of the infusion through resolution of the sedation and/or any adverse effects on blood pressure or heart rate.

Whenever the above stopping criteria was met, the site stopped the infusion and the site continued to monitor the participant's vital signs every 15 minutes until the participant has reached their baseline parameters or in the judgment of the principal investigator the participant has reached a stable and acceptable level of blood pressure and heart rate. Return to baseline parameters is defined as BP falling within 15 mm of Hg of baseline reading prior to drug administration or HR falling within 10 beats per minute of baseline reading prior to drug administration.

In the event the investigator deemed the fall in blood pressure or heart rate to be clinically significant, suitable remedial drugs could be administered in addition to termination of the dexmedetomidine hydrochloride infusion, based on investigator's judgement.

Adverse events (AEs), including serious adverse events (SAEs), were assessed, recorded, and reported in accordance with FDA guidance. Should any SAE occur, the study would be stopped until a cause for the SAE was determined.

### Efficacy Assessment

(1) Richmond Agitation Sedation Scale (RASS): The desired endpoint was how rapidly drowsiness (RASS −1) could be achieved without causing changes in heart rate or blood pressure greater than that specified by the protocol. The study also monitored how long the participant remained at that level of sedation; sedation was considered resolved when the participant was awake and spontaneously responding.

(2) PANS S: Change from Baseline for mildly agitated patients

(3) Clinical Global Impression of Improvement (CGI-I) (National Institute of Mental Health 1976) ranging from 1 (very much improved) to 7 (very much worse) compared with baseline. Each participant was rated, based on the severity of agitation, at 15 and 30 minutes for every dose infusion, at the endpoint, and at the time the participant returned to baseline (in terms of level of arousal). CGI-I focused on the severity of agitation rather than the severity of the illness.

(4) After the infusion was stopped, the participants were judged for the suitability for discharge by the principal investigator or designee as witnessed by a return to their baseline level of alertness and awareness with no impairment in balance, gait, and reaction time as determined by the principal investigator or designee.

### Results

### (A) Efficacy Study

### RASS (Richmond Agitation-Sedation Scale)

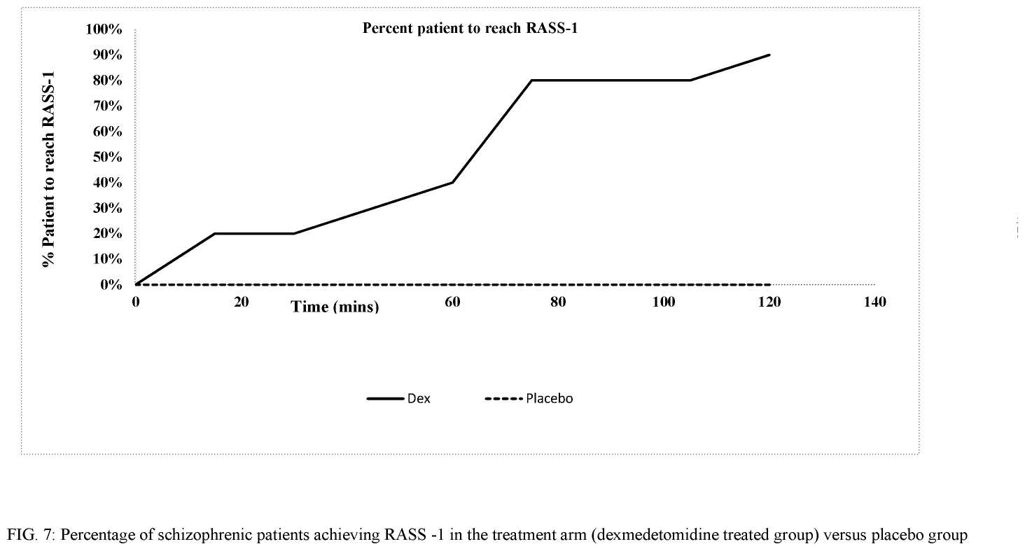

9 out of 10 patients in the treatment arm (subjects 1-3, 11, 12, 14, and 16-18) achieved a RASS score of at least −1, while no patients in the placebo arm (subjects 5, and 7-9) experienced meaningful sedation (see FIG. 7 and Table 12).

TABLE 12
Depicts the RASS score of Schizophrenia patients receiving
infusion of dexmedetomidine hydrochloride and normal saline

| | RASS values after infusion start | | | | | | | | | | | | | |
|---|---|---|---|---|---|---|---|---|---|---|---|---|---|---|
| | Patient No. | | | | | | | | | | | | | |
| Infusion | 1 | 2 | 3 | 4 | 5 | 7 | 8 | 9 | 11 | 12 | 14 | 16 | 17 | 18 |
| (minutes) | T | T | T | T | P | P | P | P | T | T | T | T | T | T |
| 0 | 1 | 3 | 1 | 1 | 1 | 1 | 1 | 1 | 1 | 1 | 1 | 1 | 1 | 0 |
| 15 | −2 | | | | | | | | | | | | | −1 |
| 30 | | 0 | 0 | 0 | 1 | 1 | 1 | 1 | 0 | 0 | 0 | 0 | 0 | |
| 45 | | | 0 | | | | | | | | | | −1 | |
| 60 | | 0 | −1 | 0 | 1 | 1 | 0 | 1 | 0 | 0 | 0 | 0 | | |
| 75 | | −1 | | | | | | | −1 | | −1 | −1 | | |
| 90 | | | | 0 | 1 | 0 | 0 | 1 | | 0 | | | | |
| 105 | | | | | | | | | | | | | | |
| 120 | | | | 0 | 0 | 0 | 0 | 1 | | −1 | | | | |
| 135 | | | | 0 | | | | | | | | | | |
| 150 | | | | | 0 | 0 | 0 | 1 | | | | | | |
| 165 | | | | | | | | | | | | | | |
| 180 | | | | | 0 | 0 | 0 | 1 | | | | | | |

T—treatment arm;
P—placebo arm

### PEC (PANSS Excitement Component)

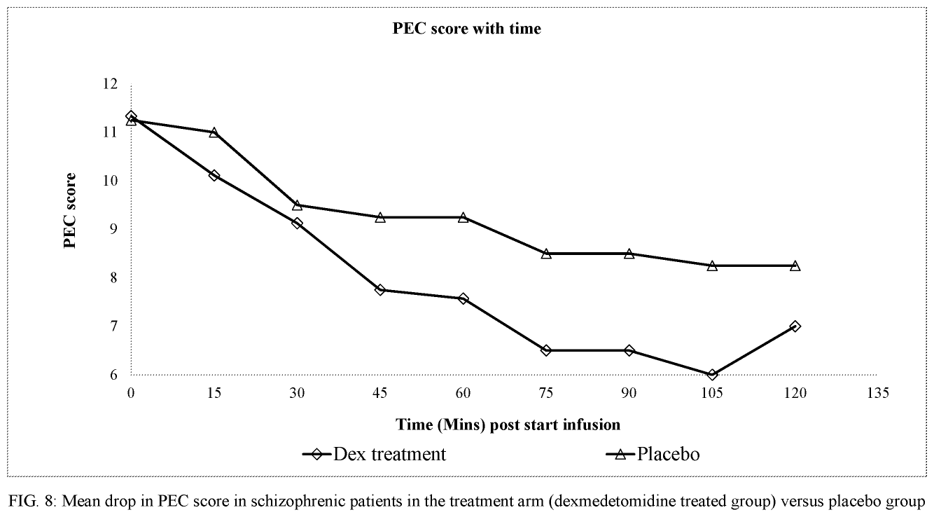

9 out of 10 patients in the treatment arm (subjects 1-4, 11, 12, 14, 16 and 17) had agitation reduced to a minimum (as measured by a PEC score of 7 or below) (see Table 13 and FIG. 8).

TABLE 13
Depicts the PEC data of schizophrenia patients receiving infusion
of dexmedetomidine hydrochloride and normal saline

| | PEC values after infusion start | | | | | | | | | | | | | |
|---|---|---|---|---|---|---|---|---|---|---|---|---|---|---|
| | Patient No. | | | | | | | | | | | | | |
| | 1 | 2 | 3 | 4 | 5 | 7 | 8 | 9 | 11 | 12 | 14 | 16 | 17 | 18 |
| Time (Mins) | T | T | T | T | P | P | P | P | T | T | T | T | T | T |
| 0 | 9 | 16 | 12 | 9 | 11 | 12 | 9 | 13 | 13 | 13 | 10 | 10 | 10 | |
| 15 | 5 | 13 | 12 | 9 | 10 | 12 | 9 | 13 | 13 | 13 | 9 | 9 | 8 | |
| 30 | | 12 | 10 | 8 | 9 | 9 | 8 | 12 | 11 | 13 | 6 | 6 | 7 | |
| 45 | | 11 | 7 | 8 | 9 | 8 | 8 | 12 | 9 | 10 | 6 | 6 | 5 | |
| 60 | | 9 | 6 | 7 | 8 | 8 | 8 | 13 | 9 | 10 | 5 | 7 | | |
| 75 | | 7 | | 7 | 8 | 8 | 7 | 11 | 7 | 8 | 5 | 5 | | |
| 90 | | | | 7 | 7 | 9 | 7 | 11 | | 6 | | | | |
| 105 | | | | 7 | 8 | 8 | 7 | 10 | | 5 | | | | |
| 120 | | | | 7 | 8 | 8 | 7 | 10 | | | | | | |
| 135 | | | | 7 | 8 | 7 | 7 | 9 | | | | | | |
| 150 | | | | | 8 | 7 | 7 | 9 | | | | | | |
| 165 | | | | | 8 | 8 | 7 | 9 | | | | | | |
| 180 | | | | | 8 | 8 | 7 | 10 | | | | | | |

T—treatment arm;
P—placebo arm

### (B) Pharmacokinetic Study: (PK Study)

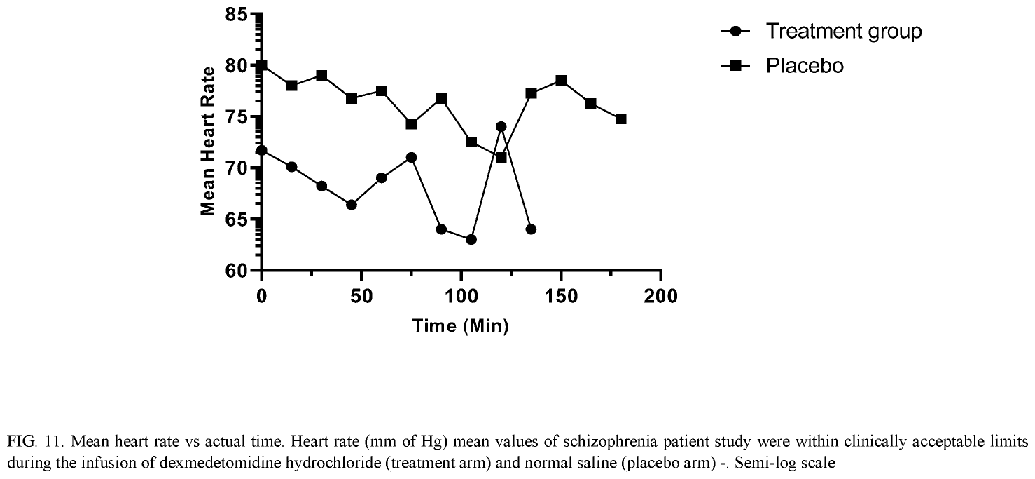

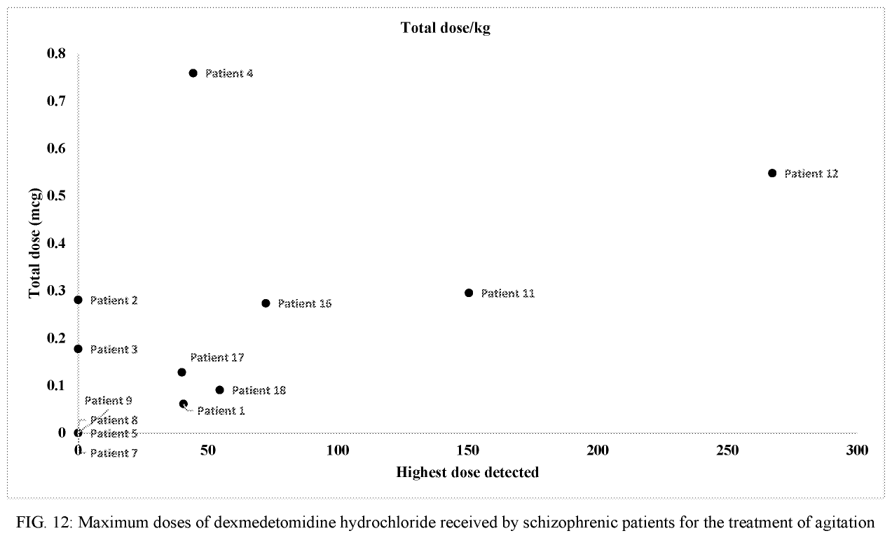

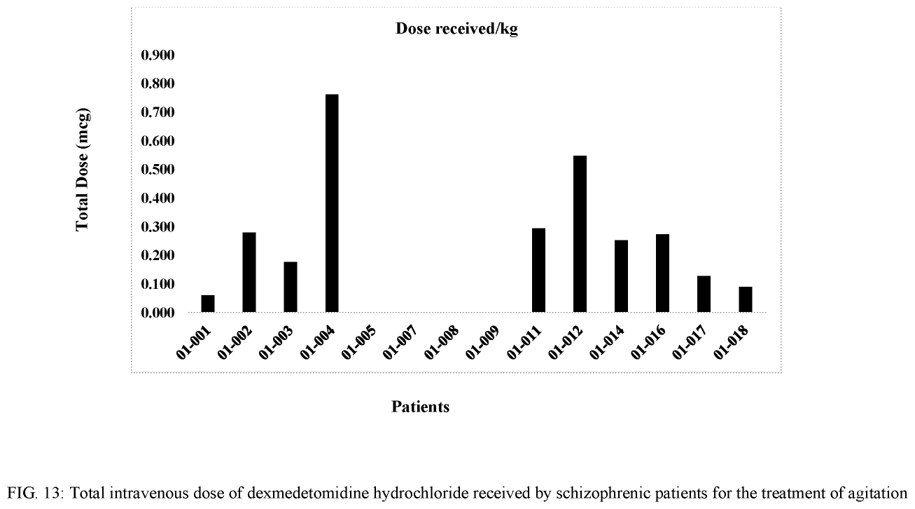

The level of dexmedetomidine in the plasma of patients was also measured over the time of infusion. The results are tabulated in Table 14. The maximum dexmedetomidine concentrations in schizophrenic patients (Cₘₐₓ) ranged from about 22.45 pg/ml to about 406.3 pg/ml. Time to reach Cₘₐₓ ranged from about 15 minutes to about 105 minutes. Mean infusion rate is 0.36 mcg/kg/hr with the maximum rate ranging from about 0.2 mcg/kg/hr to about 0.6 mcg/kg/hr (see FIGS. 11, 12 and 13).

TABLE 14
depicts the plasma concentrations (pg/mL) of schizophrenia patients at different
timepoints during the infusion of dexmedetomidine hydrochloride and normal saline

| | Plasma level concentration (picogram/ml) | | | | | | | | | | | | | |
|---|---|---|---|---|---|---|---|---|---|---|---|---|---|---|
| | 1 | 2 | 3 | 4 | 5 | 7 | 8 | 9 | 11 | 12 | 14 | 16 | 17 | 18 |
| Time (Mins) | T | T | T | T | P | P | P | P | T | T | T | T | T | T |
| 0 | BLQ | BLQ | | BLQ | BLQ | BLQ | BLQ | BLQ | BLQ | BLQ | BLQ | BLQ | BLQ | BLQ |
| 15 | 22.45 | | | BLQ | BLQ | BLQ | BLQ | BLQ | 41.01 | BLQ | BLQ | 2.56 | 15.87 | 48.36 |
| 30 | 14.72 | BLQ | BLQ | BLQ | BLQ | BLQ | BLQ | BLQ | 62.91 | 44.87 | 52.66 | 15.59 | BLQ | 54.53 |
| 45 | | | BLQ | BLQ | BLQ | BLQ | BLQ | BLQ | 124.07 | 50.51 | 46.53 | 41.17 | 39.93 | |
| 60 | | | | BLQ | BLQ | BLQ | BLQ | BLQ | 150.47 | 108.6 | 406.3 | 67.88 | | |
| 75 | | | | BLQ | BLQ | BLQ | BLQ | BLQ | | 158.54 | | 72.26 | | |
| 90 | | | | 44.3 | BLQ | BLQ | BLQ | BLQ | | 237.83 | | | | |
| 105 | | | | | BLQ | BLQ | BLQ | BLQ | | 267.3 | | | | |
| 120 | | | | | BLQ | BLQ | BLQ | BLQ | | | | | | |
| 135 | | | | | BLQ | BLQ | BLQ | BLQ | | | | | | |
| 150 | | | | | BLQ | BLQ | BLQ | BLQ | | | | | | |
| 165 | | | | | BLQ | BLQ | BLQ | BLQ | | | | | | |
| 180 | | | | | BLQ | BLQ | BLQ | BLQ | | | | | | |
| Total duration | 19 | 75 | 60 | 149 | 180 | 180 | 180 | 179 | 68 | 103 | 64 | 66 | 36 | 30 |
| of infusion | | | | | | | | | | | | | | |
| (Mins) | | | | | | | | | | | | | | |

*BLQ—below limit of quantification
T—Treatment;
P—Placebo

### (C) Safety Study

TABLE 15
Adverse effect (AE) - one subject

| AE Term | Severity | Relationship | Outcome |
|---|---|---|---|
| Headache | Moderate | Possibly Related | Recovered |

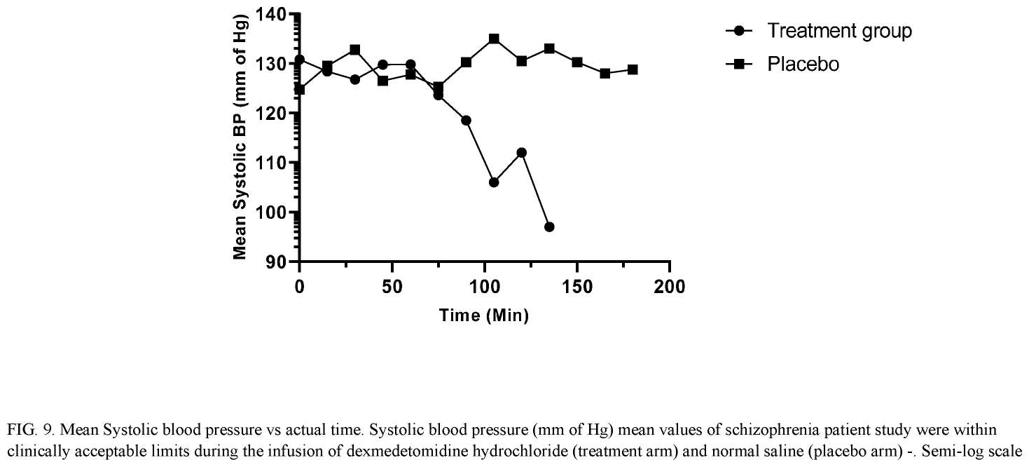

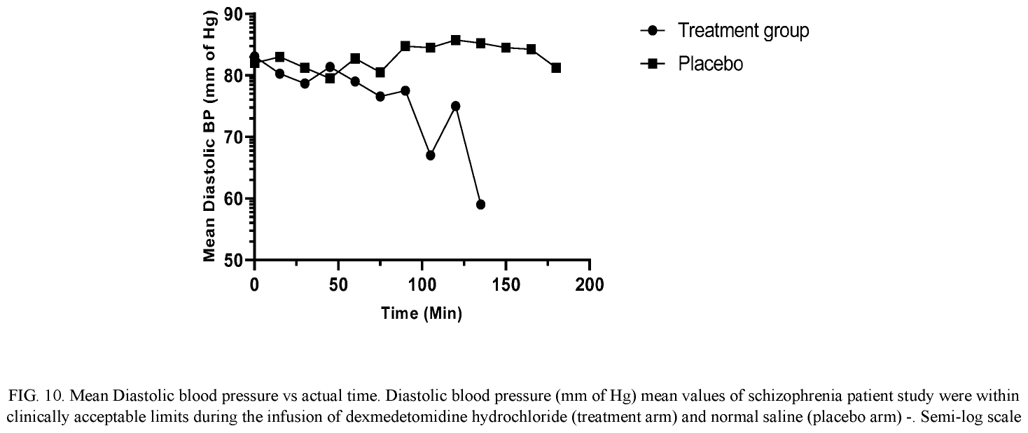

Dexmedetomidine hydrochloride infusion in schizophrenic patients showed no adverse effect on hematological parameters such as blood pressure (systolic and diastolic) and heart rate. The results were illustrated in FIGS. 9, 10 and 11 and also tabulated in tables 16A, 16B and 16C. Patient no. 4 met the stopping criteria of <60 mm Hg after 135 minutes of dexmedetomidine hydrochloride infusion.

Systolic blood pressure (mm of Hg) of Schizophrenic patients: Systolic pressure of patients with time after administering dexmedetomidine hydrochloride infusion in schizophrenic patients (see Table 16A)

TABLE 16A

| | | | Systolic blood pressure (mm of Hg) | | | | | |
|---|---|---|---|---|---|---|---|---|
| | | | 00:00 | 00:15 | 00:30 | 00:45 | 01:00 | 01:15 |
| Group | Statistics | Screening | Hr | Hr | Hr | Hr | Hr | Hr |
| Dex | N | 10 | 10 | 10 | 9 | 8 | 6 | 5 |
| | Mean ± SD | 126 ± 9.72 | 130.8 ± 11.84 | 128.4 ± 15.98 | 126.77 ± 14.68 | 129.75 ± 12.64 | 129.83 ± 17.56 | 123.6 ± 13.72 |
| | Median | 124 | 131 | 127 | 129 | 129 | 124 | 119 |
| | Min, Max | 113, 141 | 110, 148 | 101, 162 | 105, 155 | 106, 150 | 117, 168 | 108, 149 |
| Placebo | N | 4 | 4 | 4 | 4 | 4 | 4 | 4 |
| | Mean ± SD | 121.25 ± 13.06 | 124.75 ± 13.51 | 129.5 ± 20.65 | 132.75 ± 27.04 | 126.5 ± 16.08 | 127.75 ± 15.94 | 125.25 ± 18.23 |
| | Median | 119.5 | 127 | 126 | 125.5 | 124 | 121.5 | 128 |
| | Min, Max | 108, 138 | 105, 140 | 106, 160 | 105, 175 | 108, 150 | 114, 154 | 100, 145 |

| | | Systolic blood pressure (mm of Hg) | | | | | | |
|---|---|---|---|---|---|---|---|---|
| | | 01:30 | 01:45 | 02:00 | 02:15 | 02:30 | 02:45 | 03:00 |
| Group | Statistics | Hr | Hr | Hr | Hr | Hr | Hr | Hr |
| Dex | N | 2 | 1 | 1 | 1 | | | |
| | Mean ± SD | 118.5 ± 5.5 | 106 | 112 | 97 | | | |
| | Median | 118.5 | 106 | 112 | 97 | | | |
| | Min, Max | 113, 124 | | | | | | |
| Placebo | N | 4 | 4 | 4 | 4 | 4 | 4 | 4 |
| | Mean ± SD | 130.25 ± 15.48 | 135 ± 15.26 | 130.5 ± 13.95 | 133 ± 16.89 | 130.25 ± 13.97 | 128 ± 13.8 | 128.75 ± 12.17 |
| | Median | 125 | 133 | 128 | 137.5 | 132 | 129.5 | 131.5 |
| | Min, Max | 115, 156 | 120, 154 | 114, 152 | 108, 149 | 113, 144 | 109, 144 | 111, 141 |

### Diastolic Blood Pressure (Mm of Hg) of Schizophrenic Patients:

Diastolic blood pressure of patients with time after administering dexmedetomidine hydrochloride infusion in schizophrenic patients (see Table 16B)

TABLE 16B

| | | | Diastolic blood pressure (mm of Hg) | | | | | |
|---|---|---|---|---|---|---|---|---|
| | | | 00:00 | 00:15 | 00:30 | 00:45 | 01:00 | 01:15 |
| Group | Statisties | Screening | Hr | Hr | Hr | Hr | Hr | Hr |
| Dex | N | 10 | 10 | 10 | 9 | 8 | 6 | 5 |
| | Mean ± SD | 82.5 ± 6.31 | 83.1 ± 6.97 | 80.3 ± 6.43 | 78.666 ± 10.6 | 81.375 ± 9.53 | 79 ± 4.93 | 76.6 ± 10.36 |
| | Median | 82 | 84.5 | 80.5 | 80 | 80.5 | 79 | 77 |
| | Min, Max | 73, 95 | 72, 92 | 68, 90 | 64, 91 | 63, 98 | 73, 85 | 63, 92 |
| Placebo | N | 4 | 4 | 4 | 4 | 4 | 4 | 4 |
| | Mean ± SD | 79.5 ± 4.55 | 82 ± 6.96 | 83 ± 4.94 | 81.25 ± 8.34 | 79.5 ± 10.11 | 82.75 ± 10.32 | 80.5 ± 10.35 |
| | Median | 81 | 85.5 | 83 | 80 | 81 | 84 | 81.5 |
| | Min, Max | 72, 84 | 70, 87 | 76, 90 | 71, 94 | 64, 92 | 67, 96 | 65, 94 |

| | | Diastolic blood pressure (mm of Hg) | | | | | | |
|---|---|---|---|---|---|---|---|---|
| | | 01:30 | 01:45 | 02:00 | 02:15 | 02:30 | 02:45 | 03:00 |
| Group | Statisties | Hr | Hr | Hr | Hr | Hr | Hr | Hr |
| Dex | N | 2 | 1 | 1 | 1 | | | |
| | Mean ± SD | 77.5 ± 5.5 | 67 | 75 | 59 | | | |
| | Median | 77.5 | 67 | 75 | 59 | NA | NA | NA |
| | Min, Max | 72, 83 | NA | NA | NA | NA | NA | NA |
| Placebo | N | 4 | 4 | 4 | 4 | 4 | 4 | 4 |
| | Mean ± SD | 84.75 ± 9.60 | 84.5 ± 9.55 | 85.75 ± 7.98 | 85.25 ± 7.72 | 84.5 ± 8.95 | 84.25 ± 8.69 | 81.25 ± 6.90 |
| | Median | 84 | 84.5 | 85.5 | 89 | 84 | 83 | 82 |
| | Min, Max | 72, 99 | 71, 98 | 75, 97 | 72, 91 | 73, 97 | 74, 97 | 71, 90 |

Heart rate (mm Hg) mean values in schizophrenic patients. Heart rate of patients with time after administration of dexmedetomidine hydrochloride and normal saline (see Table 16C)

TABLE 16C

| | | | Ventricular Heart rate (VHR) | | | | | |
|---|---|---|---|---|---|---|---|---|
| | | | 00:00 | 00:15 | 00:30 | 00:45 | 01:00 | 01:15 |
| Group | Statistics | Sereening | Hr | Hr | Hr | Hr | Hr | Hr |
| Dex | N | 10 | 10 | 10 | 9 | 8 | 6 | 5 |
| | Mean ± SD | 75.44 ± 10.03 | 71.7 ± 10.00 | 70.1 ± 9.256 | 68.222 ± 8.283 | 66.375 ± 11.24 | 69 ± 11.59 | 71 ± 9.143 |
| | Median | 77.5 | 73 | 72.5 | 69 | 67.5 | 68 | 66 |
| | Min, Max | 58, 90 | 55, 86 | 57, 84 | 55, 80 | 51, 85 | 53, 84 | 61, 83 |
| Placebo | N | 4 | 4 | 4 | 4 | 4 | 4 | 4 |
| | Mean ± SD | 75.5 ± 13.44 | 80 ± 17.49 | 78 ± 17.07 | 79 ± 19.68 | 76.75 ± 18.53 | 77.5 ± 20.18 | 74.25 ± 14.30 |
| | Median | 79 | 81 | 73 | 74 | 71.5 | 72 | 73 |
| | Min, Max | 56, 88 | 58, 100 | 61, 105 | 58, 110 | 58, 106 | 56, 110 | 58, 93 |

| | | Ventricular Heart rate (VHR) | | | | | | |
|---|---|---|---|---|---|---|---|---|
| | | 01:30 | 01:45 | 02:00 | 02:15 | 02:30 | 02:45 | 03:00 |
| Group | Statistics | Hr | Hr | Hr | Hr | Hr | Hr | Hr |
| Dex | N | 2 | 1 | 1 | 1 | 0 | 0 | 0 |
| | Mean ± SD | 64 ± 3 | 63 | 74 | 64 | NA | NA | NA |
| | Median | 64 | 63 | 74 | 64 | NA | NA | NA |
| | Min, Max | 61, 67 | NA | NA | NA | NA | NA | NA |
| Placebo | N | 4 | 4 | 4 | 4 | 4 | 4 | 4 |
| | Mean ± SD | 76.75 ± 22.55 | 72.5 ± 17.79 | 71 ± 19.33 | 77.25 ± 14.41 | 78.5 ± 14.22 | 76.25 ± 13.00 | 74.75 ± 10.03 |
| | Median | 71 | 68 | 65 | 82 | 80.5 | 78.5 | 76 |
| | Min, Max | 53, 112 | 54, 100 | 53, 101 | 55, 90 | 59, 94 | 58, 90 | 60, 87 |

### Example 3

IV Dexmedetomidine Hydrochloride in Subjects Suffering from Opiate Withdrawal Symptoms

### Primary Objective:

Primary objective was to determine the optimal intravenous (IV) dose of dexmedetomidine hydrochloride in the target population in terms of efficacy and safety to achieve 50% reduction in COWS scores.

Secondary objectives were to determine:
1. CGI change from baseline
2. how rapidly the drug was administered up to total dose needed to achieve a reduction in COWS total score.
3. how long the calming effect persists after discontinuation of study drug administration.
4. whether any adverse effects on blood pressure, heart rate, or respiratory drive occurs before or coincident with the achievement of the aforementioned reduction of COWS scale.

Stopping rules for blood pressure and heart rate are:
a. drop in systolic BP<90 mm of Hg.
b. drop in diastolic BP<60 mm of Hg
c. drop below 50 beats per minute

This was a phase I, randomized, placebo-controlled trial of the effectiveness of dexmedetomidine hydrochloride administered intravenously for the treatment of mild to moderate withdrawal symptoms in patients with opioid use disorder.

Clinical Evaluation Performance

All participants were screened for inclusion and exclusion criteria at screening. The participants were admitted to the site of the day on screening and after 8-12 hours discontinuation of opiates in order to achieve mild to moderate withdrawal. Baseline assessments were performed on the day of admission (Screening), as well as on the day of infusion (Day 1), which was followed by preparation for infusion, the infusion (up to 3 hours) and time necessary for resolution of withdrawal symptoms as defined as a 50% reduction in COWS total score and any decreases in blood pressure or heart rate which meet stopping criteria. A second infusion was given on the next day. The stopping criteria and assessment scheduled remained the same for the second infusion as for the initial infusion. The participant received standard of care detoxification treatment for the additional withdrawal symptoms, for a total of up to 4 inpatient nights. The participants were discharged from the research unit once acute withdrawal symptoms ceased and the principal investigator determined the participants had returned to a safe baseline state.

Based on projections from previous experience, this study took approximately 1 month to complete. The cohort consisted of 16 participants, 11 treated with dexmedetomidine and 5 treated with placebo.

The participants were continuously monitored by the site personnel, including blood pressure and heart rate, from the start of the infusion through resolution of sedation and/or any adverse effects on blood pressure or heart rate. An electrocardiogram was obtained at screening, pre-infusion and upon resolution of sedation, a predetermined reduction in COWS or return to baseline.

In addition to the above attainment of a 50% reduction in COWS total score, dosing was stopped for safety reasons at any time if any of the following occurred:
a. drop in systolic BP<90 mm of Hg.
b. drop in diastolic BP<60 mm of Hg
c. drop in heart rate below 50 beats per minute
d. RASS score of −1

Whenever the above stopping criteria was met, whether because of the COWS scores, BP/HR, or sedation, the infusion was stopped the participant's vital signs continued to be every 15 minutes until the participant reached their baseline parameters or, in the judgment of the principal investigator, the participant reached a stable and acceptable level of blood pressure and heart rate. Return to baseline parameters was defined as BP falling within 15 mmHg of the baseline reading prior to drug administration or HR falling within 10 beats per minute of the baseline reading prior to drug administration.

### Schedule of Assessments

TABLE 17
depicts the schedule of assessment of opioid withdrawal study

| | Screening | Baseline | Follow-Up |
|---|---|---|---|
| Activity | Day −1 | Day 1 | Day 2-4 |
| Informed Consent | X | | |
| Signed | | | |
| Subject Number | X | | |
| Assigned | | | |
| Inclusion/Exclusion | X | X (a) | |
| Criteria | | | |
| Prior Medication History | Past 30 days | X (a) | |
| Demographics | X | | |
| Medical and Smoking | X | | |
| History | | | |
| Pregnancy Test | X | X | |
| Height | X | | |
| Weight | X | | |
| Complete Physical Exam | X | X (a) | X |
| Admission to In-clinic | X | | |
| Facility (i) | | | |
| Study Medication | | X (j) | |
| Administration (j) | | | |
| Discharge from In-clinic | | | X (h) |
| Facility | | | |
| 12-Lead | X | X (c) | X (1x/day) |
| Electrocardiogram | | | |
| Urine Drug Screen | X | X | |
| Vital Signs | X | X (b) | X (4x/day) |
| (Sitting/Recumbent BP | | | |
| and pulse; respiration; | | | |
| and temperature) (b) | | | |
| Clinical Laboratory | X | As needed | As needed |
| Tests (hematology, | | | |
| chemistry, urinalysis) | | | |
| Adverse Events | X | X | X |
| Assessment | | | |
| Suicide Safety | X | X | X |
| Assessment | | | |
| Clinical Opiate | X | X(e) | X (As needed) |
| Withdrawal Scale | | | |
| (COWS) | | | |
| Clinical Global | | X (g) | |
| Impression of | | | |
| Improvement (CGI-I) | | | |
| Pharmacokinetic | | X | |
| Samples (d) | | | |
| Standard of Care | | As needed | As needed |
| Treatment | | | |
| Concomitant | X | X | X |
| Medications Assessment | | | |

Abbreviations: BP = blood pressure, PK = pharmacokinetic,
* The study discontinuation/end of study assessments/procedures should always be done when subject exits from the study.
(a) This form is to be updated at Baseline.
(b) Vital signs collected in supine position at Screening, Day 1 pre-dose and every 15 minutes through duration of infusion, and 30 minutes post recovery to baseline. Vital signs have +/−5 minutes window.
(c) 12-lead ECGs (duplicate) before dosing on Day 1 and 30 minutes post recovery to baseline (±15 minutes) after infusion stop.
(d) Pharmacokinetic samples collected on Day 1 pre-dose and every 15 minutes through duration of infusion; and 30 minutes post recovery to baseline. PK samples have +/−5 minutes window.
(e)COWS assessments collected on Day 1 pre-dose and every 15 minutes through duration of infusion; and 30 minutes post recovery to baseline. COWS assessments have +/−5 minutes window. Subjects were included in the study if they met, at Day 1, the criteria for mild-moderate withdrawal consistent with a COWS score of 9-16.
(f) RASS assessments collected on Day 1 pre-dose and every 30 minutes through duration of infusion; and 30 minutes post recovery to baseline. RASS assessments have +/−5 minutes window.
(g) CGI-I completed 30 minutes post recovery to baseline status.
(h) Discharge date dependent upon the Principle Investigator's discretion of subject safety.
(i) In-patient nights Screening, Day 1, and Days 2-4 are under discretion of the Principle Investigator for subject safety and standard of care detox.
(j) Second infusion was dependent upon the discretion of the Principle Investigator.

### Study Duration:

6-10 days including screening, one day of dexmedetomidine hydrochloride administration and second day of dexmedetomidine hydrochloride administration (4-5 patients were dosed based on discretion of the Principle Investigator) and 2-3 days post-dose assessment.

Treatments

### Study Treatments:

There were two treatment arms in the study as described below in Table 18

TABLE 18

| | | | Route of |
|---|---|---|---|
| Treatment | Formulation | Frequency | Administration |
| Dexmedetomidine | Commercial | Once | Intravenous |
| | available | (continuous | |
| | (PRECEDEX) | Infusion, | |
| | | Incremental dose | |
| | | increases every | |
| | | 30 minutes) | |
| Placebo | Normal Saline | Once (continuous | Intravenous |
| | | infusion) | |

### Treatment Administered:

The initial dose of dexmedetomidine hydrochloride was 0.2 mcg/kg/hr, with no loading dose with the desired endpoint being achieving a 50% reduction in COWS total score. Once the reduction was achieved, the infusion was stopped. The dose was increased by up to 0.1 mcg/kg/hr if the desired level of sedation was not achieved by the end of 30 minutes of infusion of the prior dose. The infusion was continued for up to 2.5 hours up to the maximum dose. In no instance was the infusion rate exceed 1.0 mcg/kg/hr and the maximum total dose administered was capped to 1.6 mcg/kg/hr for any participant.

### Statistical Methods:

### Descriptive in Terms of Total Dose and Rate of Delivery:

Tables were constructed containing the mean and standard deviations of blood pressure, heart rate, and respiratory rate at baseline, the point infusion was stopped, and at recovery from any decrease in arousal from baseline as well as maximum changes in these values at any time from the start of the infusion to the period of recovery.

### Permitted Concomitant Medications:

Any medication not explicitly excluded.

### Efficacy Assessment:

### Primary Endpoints:

### Clinical Opiate Withdrawal Scale (COWS):

The COWS was used to assess the effectiveness of dexmedetomidine hydrochloride in alleviation of opioid withdrawal symptoms, targeting agitation related items, and was completed at screening, baseline, at 15 and 30 minutes for every dose infusion, at endpoint, and at the time that participant returned to baseline in terms of level of arousal. The COWS is a clinician-administered instrument that rates 11 common opioid withdrawal signs and symptoms. These include: resting pulse rate; sweating; restlessness (agitation or hyper-arousal); pupil size; bone or joint aches; runny nose or tearing; gastrointestinal (GI) upset; tremor; yawning; anxiety or irritability; and gooseflesh skin. The score for each item reflects the severity of the sign or symptom, and the total scores are grouped as mild (5-12 points), moderate (13-24 points), moderately severe (25-36 points), and severe (>36 points).

### Secondary Endpoints:

### 1. Clinical Global Impression of Severity (CGI-S) (National Institute of Mental Health 1976):

The Clinical Global Impression of Severity scale range from 1 (normal, not at all ill) to 7 (among the most extremely ill) was rated, based upon the severity of agitation, at screening, baseline, at endpoint, and at the time that participant returned to baseline in terms of level of arousal.

### 2. Clinical Global Impression of Improvement (CGI-I) (National Institute of Mental Health 1976):

Clinical Global Impression of Improvement range from 1 (very much improved) to 7 (very much worse) compared with baseline, was rated, based on the severity of agitation, at endpoint, and at the time that participant returns to baseline in terms of level of arousal.

Both CGI-I and CGI-S focused on the severity of agitation rather than the severity of illness.

### Safety Assessments:

Continuous assessment before and during study drug infusion and through resolution of the induced sedation. The site continuously monitored the participant in terms of
1. Level of sedation as assessed by the RASS
2. Vital signs

Additional Assessment Included:
1. Hematological and blood chemistry, urinalysis
2. Physical examination
3. Standard of care detoxification treatment (ancillary medications) to alleviate symptoms of acute withdrawal to ensure safe discharge from study site.
4. Adverse event reporting

### Results

TABLE 19
Demographic and other baseline characteristics

| Demographic | | Test | | Placebo | | Overall | |
|---|---|---|---|---|---|---|---|
| Characteristics | Statistics | (N = 11) | | (N = 5) | | (N = 16) | |
| Age (Years) | N | 11 | | 5 | | 16 | |
| | Mean ± SD | 44 ± 7.24 | | 39.4 ± 9.91 | | 42.56 ± 8.44 | |
| | Median | 41 | | 41 | | 41 | |
| | Min, Max | 36, 57 | | 27, 51 | | 27, 57 | |
| Sex [n (%)] | Male (1) | 10 | (90.91) | 4 | (80.00) | 14 | (87.5) |
| | Female (2) | 1 | (09.09) | 1 | (20.00) | 2 | (12.50) |
| Race [n (%)] | White (5) | 8 | (72.72) | 4 | (80.00) | 12 | (75.00) |
| | Black or African | 3 | (27.27) | 1 | (20.00) | 4 | (25.00) |
| | American (2) | | | | | | |
| | American Indian | 0 | (0.00) | 0 | (0.00) | 0 | (0.00) |
| | or Alaskan | | | | | | |
| | Native (1) | | | | | | |
| | Native Hawaiian | 0 | (0.00) | 0 | (0.00) | 0 | (0.00) |
| | or Other Pacific | | | | | | |
| | Islander (3) | | | | | | |
| | Asian (4) | 0 | (0.00) | 0 | (0.00) | 0 | (0.00) |
| Ethnicity [n (%)] | Non-Hispanic | 8 | (72.72) | 2 | (40.00) | 10 | (62.50) |
| | Hispanic | 3 | (27.27) | 3 | (60.00) | 6 | (37.50) |
| Height (cm) | N | 11 | (90.91) | 5 | (80.00) | 16 | (87.5) |

| | | | | |
|---|---|---|---|---|
| | Mean ± SD | 175.67 ± 7.12 | 171.58 ± 7.65 | 174.39 ± 7.53 |
| | Median | 173 | 170 | 172.5 |
| | Min, Max | 166.5, 188.5 | 161, 182.9 | 161, 188.5 |
| Weight (kg) | N | 11 | 5 | 16 |
| | Mean ± SD | 79.51 ± 19.89 | 74.92 ± 19.28 | 78.08 ± 19.81 |
| | Median | 74.80 | 71.60 | 73.20 |
| | Min, Max | 63.9, 136.9 | 57.2, 111.1 | 57.2, 136.9 |
| BMI (kg/m²) | N | 11 | 5 | 16 |
| | Mean ± SD | 25.95 ± 7.46 | 25.14 ± 4.45 | 25.7 ± 6.68 |
| | Median | 23.5 | 23.9 | 23.7 |
| | Min, Max | 19.9, 48.5 | 19.8, 33.2 | 19.8, 48.5 |

Abbreviations: BMI = body mass index; N = number of subjects in respective treatment; n = number of subjects in specified category;
Note:
Percentages are based on the number of subjects in the specified treatment.

### Primary Endpoint—COWS Score:

11 patients were dosed. In one patient the stopping criteria was met as the heart rate fell down to 50 or below after 15 minutes. Therefore, one patient was excluded from the results interpretation.

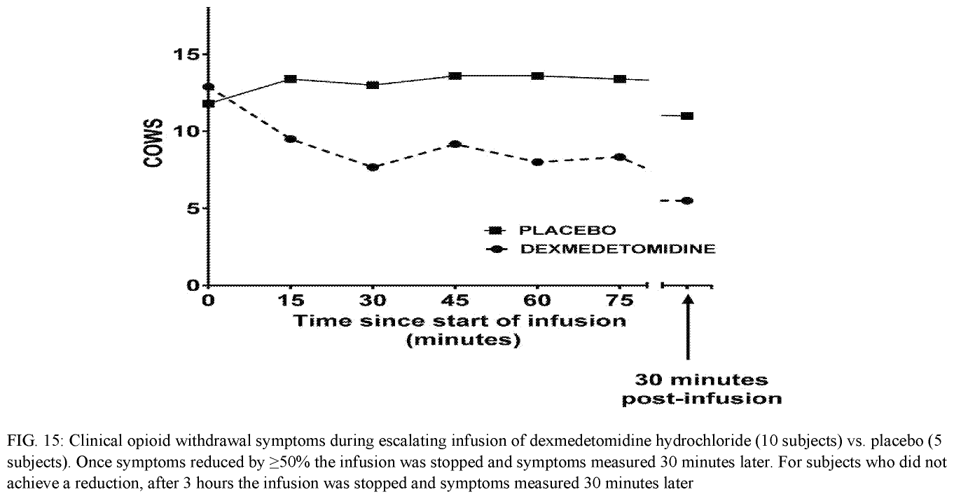

Results demonstrated that on Day 1, 10 out of 10 subjects achieved 50% reduction in COW score by 90 minutes with dexmedetomidine hydrochloride IV infusion (see Table 20 and FIG. 15), while there was no 50% reduction in COW score in patients (0/5) in the placebo arm. Further, the study demonstrated that on Day 2, 5 out of 5 patients in the treatment arm achieved 50% reduction in COW score (see Table 21). Symptoms of opioid withdrawal were evaluated using the COWS. Results from this study demonstrated that dexmedetomidine hydrochloride effectively mitigated the physiological symptoms of opioid withdrawal.

TABLE 20

| | COW score | | | | | | | | | | | | | |
|---|---|---|---|---|---|---|---|---|---|---|---|---|---|---|
| | | | | | | | | | | | | | | 30 min. |
| Patient | 00:00 | 00:15 | 00:30 | 00:45 | 01:00 | 01:15 | 01:30 | 01:45 | 02:00 | 02:15 | 02:30 | 02:45 | 03:00 | post |
| Code | Hr | Hr | Hr | Hr | Hr | Hr | Hr | Hr | Hr | Hr | Hr | Hr | Hr | Infusion |
| OP-1 | 9 | 2 | | | | | | | | | | | | 0 |
| (Treatment) | | | | | | | | | | | | | | |
| OP-2 | 15 | 11 | 9 | 12 | 2 | | | | | | | | | 16 |
| (Treatment) | | | | | | | | | | | | | | |
| OP-3 | 13 | 8 | 8 | 4 | | | | | | | | | | 3 |
| (Treatment) | | | | | | | | | | | | | | |
| OP-4 | 15 | 15 | 16 | 14 | 16 | 16 | 17 | 15 | 15 | 12 | 16 | 16 | 15 | 11 |
| (Placebo) | | | | | | | | | | | | | | |
| OP-5 | 16 | 16 | 13 | 17 | 16 | 14 | 14 | 15 | 15 | 14 | 12 | 14 | 14 | 14 |
| (Placebo) | | | | | | | | | | | | | | |
| OP-6 | 11 | 7 | 2 | | | | | | | | | | | 2 |
| (Treatment) | | | | | | | | | | | | | | |
| OP-7 | 10 | 8 | | | | | | | | | | | | 0 |
| (Treatment) | | | | | | | | | | | | | | |
| OP-8 | 9 | 10 | 10 | 11 | 12 | 11 | 10 | 11 | 13 | 11 | 10 | 10 | 8 | 8 |
| (Placebo) | | | | | | | | | | | | | | |
| OP-9 | 16 | 13 | 10 | 9 | 10 | 6 | | | | | | | | 9 |
| (Treatment) | | | | | | | | | | | | | | |
| OP-10 | 10 | 7 | 1 | | | | | | | | | | | 3 |
| (Treatment) | | | | | | | | | | | | | | |
| OP-11 | 16 | 16 | 17 | 13 | 14 | 10 | 8 | | | | | | | 8 |
| (Treatment) | | | | | | | | | | | | | | |
| OP-12 | 16 | 12 | 9 | 9 | 10 | 9 | 4 | | | | | | | 5 |
| (Treatment) | | | | | | | | | | | | | | |
| OP-13 | 9 | 10 | 10 | 10 | 10 | 11 | 10 | 10 | 11 | 12 | 11 | 11 | 12 | 12 |
| (Placebo) | | | | | | | | | | | | | | |
| OP-14 | 10 | 16 | 16 | 16 | 14 | 15 | 15 | 15 | 12 | 15 | 15 | 15 | 12 | 10 |
| (Placebo) | | | | | | | | | | | | | | |
| OP-15 | 13 | 8 | 4 | | | | | | | | | | | 4 |
| (Treatment) | | | | | | | | | | | | | | |
| OP-17 | 10 | 11 | 9 | 8 | 4 | | | | | | | | | 5 |
| (Treatment) | | | | | | | | | | | | | | |

Hr—hour

TABLE 21

| | COW score | | | | | | | | | | | | | |
|---|---|---|---|---|---|---|---|---|---|---|---|---|---|---|
| | | | | | | | | | | | | | | 30 min. |
| Patient | 00:00 | 00:15 | 00:30 | 00:45 | 01:00 | 01:15 | 01:30 | 01:45 | 02:00 | 02:15 | 02:30 | 02:45 | 03:00 | post |
| Code | Hr | Hr | Hr | Hr | Hr | Hr | Hr | Hr | Hr | Hr | Hr | Hr | Hr | Infusion |

| | | | | | | | | | | |
|---|---|---|---|---|---|---|---|---|---|---|
| OP- 1 | 9 | 8 | 2 | | | | | | | 0 |
| (Treatment) | | | | | | | | | | |
| OP-2 | 14 | 11 | 12 | 10 | 9 | 7 | | | | 4 |
| (Treatment) | | | | | | | | | | |
| OP-3 | 10 | 8 | 6 | 4 | | | | | | 4 |
| (Treatment) | | | | | | | | | | |
| OP-9 | 18 | 18 | 14 | 14 | 14 | 12 | 12 | 12 | 9 | 9 |
| (Treatment) | | | | | | | | | | |
| OP-17 | 14 | 14 | 12 | 11 | 5 | | | | | 5 |
| (Treatment) | | | | | | | | | | |

Summary of optimal dose (mcg/kg/hr) at 50% of the COW score achieved based on COW score is presented in Table 22.

TABLE 22

| Statistic | Test (Day 1) | Test (Day 2) |
|---|---|---|
| N | 10 | 5 |
| Mean ± Sd | 0.29 ± 0.08 | 0.44 ± 0.16 |
| Median | 0.3 | 0.4 |
| Min, Max | 0.2, 0.4 | 0.2, 0.7 |

Abbreviations: N = number of subjects in respective treatment group; n = number of subjects in specified category;
Note:
Subject was considered as half of the cows achieved

### Safety Evaluations

### Extent of Exposure:

### Adverse Events and Other Important Safety Findings:

No adverse event was reported during the study.

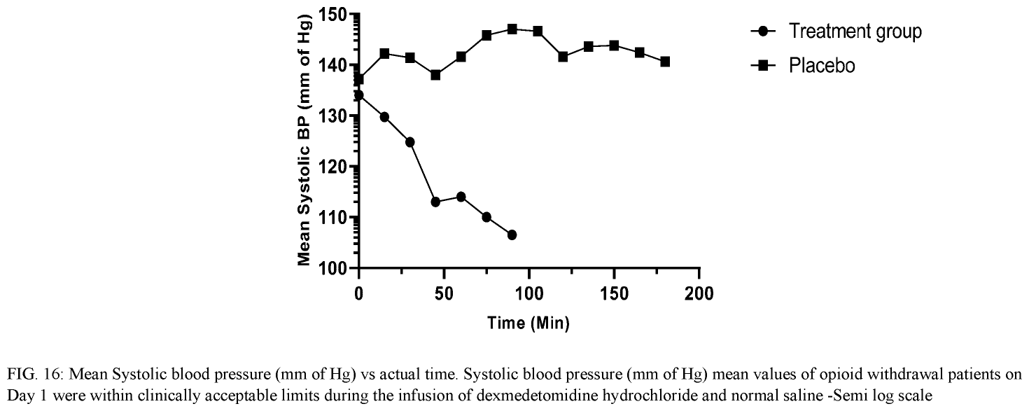

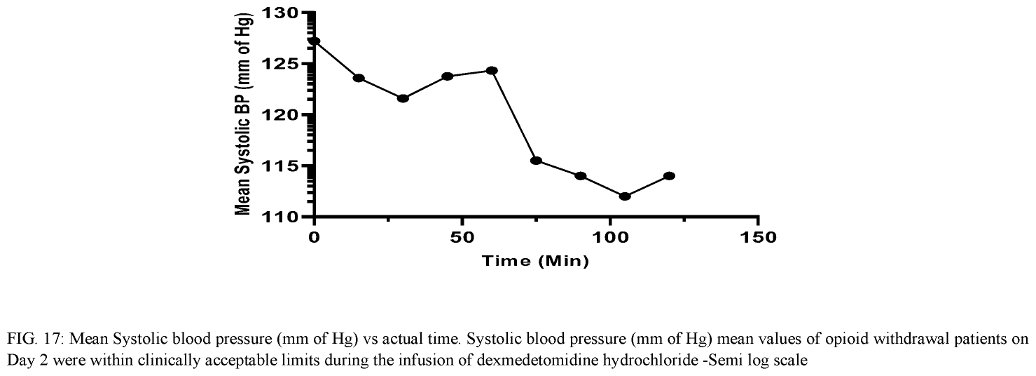

Summary of vital signs is presented in FIGS. 16, 17 18, 19, 20 and 21 and also tabulated in tables 23A, 23B and 23C. All the vital signs values were within clinically acceptable limits.

Systolic blood pressure (mm of Hg) mean values of opioid withdrawal patients were within clinically acceptable limits during the infusion of dexmedetomidine hydrochloride and normal saline (see in Table 23A)

TABLE 23A

| | | | Systolic blood pressure (mm of Hg) | | | | |
|---|---|---|---|---|---|---|---|
| Treatment | | | 00:00 | 00:15 | 00:30 | 00:45 | 01:00 |
| Group | Statistics | Screening | Hr | Hr | Hr | Hr | Hr |
| Dex | n | 11 | 11 | 11 | 9 | 6 | 5 |
| Day-1 | Mean ± SD | 130.36 ± 16.92 | 134 ± 15.77 | 129.73 ± 13.68 | 124.78 ± 15.55 | 113 ± 8.24 | 114 ± 4.85 |
| | Median | 127 | 127 | 127 | 128 | 114 | 115 |
| | Min, Max | 110, 170 | 120, 178 | 112, 157 | 107, 160 | 100, 125 | 107, 120 |
| Dex | n | 0 | 5 | 5 | 5 | 4 | 3 |
| Day-2 | Mean ± SD | 0 | 127.2 ± 11.37 | 123.6 ± 13.41 | 121.6 ± 12.15 | 123.15 ± 9.17 | 124.33 ± 9.46 |
| | Median | 0 | 133 | 131 | 125 | 128 | 130 |
| | Min, Max | 0 | 109, 141 | 103, 139 | 106, 137 | 108, 131 | 111, 132 |
| Placebo | n | 4 | 5 | 5 | 5 | 5 | 5 |
| Day-1 | Mean ± SD | 138.75 ± 5.53 | 137.2 ± 16.06 | 142.2 ± 20.51 | 141.4 ± 16.82 | 138 ± 19.11 | 141.6 ± 20.37 |
| | Median | 136.5 | 145 | 150 | 140 | 139 | 140 |
| | Min, Max | 134, 148 | 109, 152 | 110, 167 | 116, 163 | 110, 169 | 112, 166 |

| | | Systolic blood pressure (mm of Hg) | | | | |
|---|---|---|---|---|---|---|
| Treatment | | 01:15 | 01:30 | 01:45 | 02:00 | 02:15 |
| Group | Statistics | Hr | Hr | Hr | Hr | Hr |
| Dex | n | 3 | 2 | | | |
| Day-1 | Mean ± SD | 110 ± 1.63 | 106.5 ± 5.5 | | | |
| | Median | 110 | 106.5 | | | |
| | Min, Max | 108, 112 | 101, 112 | | | |
| Dex | n | 2 | 1 | 1 | 1 | |
| Day-2 | Mean ± SD | 115.5 ± 14.5 | 114 | 112 | 114 | |
| | Median | 115.5 | 114 | 112 | 114 | |
| | Min, Max | 101, 130 | | | | |
| Placebo | n | 5 | 5 | 5 | 5 | 5 |
| Day-1 | Mean ± SD | 145.8 ± 19.51 | 147 ± 17.57 | 146.6 ± 17.86 | 141.6 ± 18.26 | 143.6 ± 17.81 |
| | Median | 148 | 150 | 148 | 142 | 148 |
| | Min, Max | 116, 170 | 117, 166 | 118, 170 | 110, 166 | 112, 160 |

| | | Systolic blood pressure (mm of Hg) | | | |
|---|---|---|---|---|---|
| | | | | | 30 min. |
| Treatment | | 02:30 | 02:45 | 03:00 | post |
| Group | Statistics | Hr | Hr | Hr | Infusion |
| Dex | n | | | | 11 |
| Day-1 | Mean ± SD | | | | 130.91 ± 18.09 |
| | Median | | | | 129 |
| | Min, Max | | | | 108, 160 |
| Dex | n | | | | 5 |
| Day-2 | Mean ± SD | | | | 127.6 ± 13.1 |
| | Median | | | | 130 |
| | Min, Max | | | | 110, 146 |
| Placebo | n | 5 | 5 | 5 | 5 |
| Day-1 | Mean ± SD | 143.8 ± 19.86 | 142.4 ± 16.88 | 140.6 ± 15.33 | 137.6 ± 14.38 |
| | Median | 150 | 148 | 148 | 140 |
| | Min, Max | 109, 162 | 111, 158 | 112, 155 | 110, 150 |

Diastolic blood pressure (mm of Hg) mean values of opioid withdrawal patients were within clinically acceptable limits during the infusion of dexmedetomidine hydrochloride and normal saline (see in Table 23B)

TABLE 23B

| | | | Diastolic blood pressure (mm of Hg) | | | | |
|---|---|---|---|---|---|---|---|
| Treatment | | | 00:00 | 00:15 | 00:30 | 00:45 | 01:00 |
| group | Statistics | Screening | Hr | Hr | Hr | Hr | Hr |
| Patients | n | 11 | 11 | 11 | 9 | 6 | 5 |
| treated with | Mean ± SD | 84.18 ± 13.13 | 83.81 ± 12.99 | 78.09 ± 9.99 | 70 ± 8.8 | 74.5 ± 7.67 | 71.4 ± 6.05 |
| Dexmedetomidine | Median | 81 | 82 | 76 | 68 | 76 | 72 |
| Day-1 | Min, Max | 63, 108 | 64, 110 | 62, 96 | 59, 86 | 61, 86 | 60, 77 |
| Patients | n | 0 | 5 | 5 | 5 | 4 | 3 |
| treated with | Mean ± SD | 0 | 78.4 ± 7.86 | 77.4 ± 9.91 | 73.4 ± 8.54 | 79 ± 7.68 | 79.33 ± 7.58 |
| Dexmedetomidine | Median | 0 | 79 | 76 | 71 | 82 | 82 |
| Day-2 | Min, Max | 0 | 69, 88 | 62, 90 | 61, 84 | 66, 86 | 69, 87 |
| Placebo | N | 4 | 5 | 5 | 5 | 5 | 5 |
| Day-1 | Mean ± SD | 86 ± 4.94 | 80 ± 10.37 | 82.4 ± 10.3 | 80.4 ± 14.34 | 76.2 ± 14.03 | 87.4 ± 20.52 |
| | Median | 85.5 | 80 | 82 | 86 | 77 | 84 |
| | Min, Max | 80, 93 | 67, 93 | 70, 94 | 60, 96 | 62, 100 | 66, 120 |

| | | Diastolic blood pressure (mm of Hg) | | | | |
|---|---|---|---|---|---|---|
| Treatment | | 01:15 | 01:30 | 01:45 | 02:00 | 02:15 |
| group | Statistics | Hr | Hr | Hr | Hr | Hr |
| Patients | n | 3 | 2 | | | |
| treated with | Mean ± SD | 72 ± 8.16 | 61 ± 5 | | | |
| Dexmedetomidine | Median | 72 | 61 | | | |
| Day-1 | Min, Max | 62, 82 | 56, 66 | | | |
| Patients | n | 2 | 1 | 1 | 1 | 0 |
| treated with | Mean ± SD | 78.5 ± 22.5 | 75 | 89 | 90 | NA |
| Dexmedetomidine | Median | 78.5 | 75 | 89 | 90 | NA |
| Day-2 | Min, Max | 56, 101 | 75 | 89 | 90 | NA |
| Placebo | N | 5 | 5 | 5 | 5 | 5 |
| Day-1 | Mean ± SD | 88.6 ± 18.62 | 86.8 ± 15.97 | 84.6 ± 13.67 | 82.8 ± 13.28 | 84.4 ± 14.77 |
| | Median | 86 | 88 | 86 | 84 | 84 |
| | Min, Max | 67, 110 | 66, 108 | 68, 100 | 66, 101 | 68, 102 |

| | | Diastolic blood pressure (mm of Hg) | | | |
|---|---|---|---|---|---|
| | | | | | 30 min. |
| Treatment | | 02:30 | 02:45 | 03:00 | post |
| group | Statistics | Hr | Hr | Hr | Infusion |
| Patients | n | | | | 11 |
| treated with | Mean ± SD | | | | 75.27 ± 9.22 |
| Dexmedetomidine | Median | | | | 73 |
| Day-1 | Min, Max | | | | 60, 86 |
| Patients | n | 0 | 0 | 0 | 5 |
| treated with | Mean ± SD | NA | NA | NA | 84.4 ± 9.24 |
| Dexmedetomidine | Median | NA | NA | NA | 86 |
| Day-2 | Min, Max | NA | NA | NA | 70, 98 |
| Placebo | N | 5 | 5 | 5 | 5 |
| Day-1 | Mean ± SD | 84.8 ± 13.89 | 84.6 ± 11.89 | 81.6 ± 9.52 | 80.8 ± 8.54 |
| | Median | 86 | 86 | 84 | 82 |
| | Min, Max | 68, 100 | 70, 98 | 69, 92 | 70, 90 |

Heart rate (beats/minutes) mean values of opioid withdrawal patients were within clinically acceptable limits during the infusion of dexmedetomidine hydrochloride and normal saline (see in Table 23C)

TABLE 23C

| | | | Heart rate(beats/minutes) | | | | |
|---|---|---|---|---|---|---|---|
| | | | 00:00 | 00:15 | 00:30 | 00:45 | 01:00 |
| Treatment group | Statistics | Screening | Hr | Hr | Hr | Hr | Hr |
| Patients | n | 11 | 11 | 11 | 9 | 6 | 5 |
| treated with | Mean ± SD | 68.54 ± 15.32 | 69.9 ± 16.37 | 66.36 ± 11.9 | 66.88 ± 12.26 | 68.83 ± 14.91 | 69.6 ± 11.35 |
| Dexmedetomidine | Median | 66 | 66 | 67 | 70 | 66 | 66 |
| Day-1 | Min. Max | 55, 108 | 55, 106 | 49, 86 | 52, 84 | 50, 89 | 54, 85 |
| Patients | n | 0 | 5 | 5 | 5 | 4 | 3 |
| treated with | Mean ± SD | 0 | 68.8 ± 13.94 | 67.4 ± 12.35 | 68.2 ± 5.54 | 69 ± 16.80 | 72.66 ± 17.15 |
| Dexmedetomidine | Median | 0 | 63 | 61 | 58 | 63 | 72 |
| Day-2 | Min, Max | 0 | 55, 94 | 53, 85 | 54, 93 | 53, 97 | 52, 94 |
| Placebo | n | 4 | 5 | 5 | 5 | 5 | 5 |
| Day-1 | Mean ± SD | 67.5 ± 2.06 | 68 ± 5.69 | 65 ± 7.58 | 66.4 ± 6.34 | 64 ± 5.69 | 63.4 ± 5.91 |
| | Median | 67.5 | 66 | 63 | 70 | 66 | 64 |
| | Min, Max | 65, 70 | 60, 77 | 56, 78 | 54, 71 | 55, 70 | 53, 70 |

| | | Heart rate(beats/minutes) | | | | |
|---|---|---|---|---|---|---|
| | | 01:15 | 01:30 | 01:45 | 02:00 | 02:15 |
| Treatment group | Statistics | Hr | Hr | Hr | Hr | Hr |
| Patients | n | 3 | 2 | | | |
| treated with | Mean ± SD | 72 ± 2.82 | 60.5 ± 0.5 | | | |
| Dexmedetomidine | Median | 70 | 60.5 | | | |
| Day-1 | Min. Max | 70, 76 | 60, 61 | | | |
| Patients | n | 2 | 1 | 1 | 1 | 0 |
| treated with | Mean ± SD | 88 ± 12 | 73 | 92 | 94 | NA |
| Dexmedetomidine | Median | 88 | 73 | 92 | 94 | NA |
| Day-2 | Min, Max | 76, 100 | 73 | 92 | 94 | 0 |
| Placebo | n | 5 | 5 | 5 | 5 | 5 |
| Day-1 | Mean ± SD | 65.4 ± 5.78 | 66 ± 6.19 | 67.8 ± 6.33 | 66.4 ± 6.85 | 66.4 ± 3.26 |
| | Median | 66 | 70 | 70 | 70 | 65 |
| | Min, Max | 36, 74 | 34, 70 | 36, 74 | 34, 72 | 63, 72 |

| | | Heart rate(beats/minutes) | | | |
|---|---|---|---|---|---|
| | | | | | 30 min. |
| | | 02:30 | 02:45 | 03:00 | post |
| Treatment group | Statistics | Hr | Hr | Hr | Infusion |
| Patients | n | | | | 11 |
| treated with | Mean ± SD | | | | 65.18 ± 14.03 |
| Dexmedetomidine | Median | | | | 62 |
| Day-1 | Min. Max | | | | 50, 99 |
| Patients | n | 0 | 0 | 0 | 5 |
| treated with | Mean ± SD | NA | NA | NA | 70.8 ± 15.72 |
| Dexmedetomidine | Median | NA | NA | NA | 60 |
| Day-2 | Min, Max | 0 | 0 | 0 | 56, 90 |
| Placebo | n | 5 | 5 | 5 | 5 |
| Day-1 | Mean ± SD | 68.6 ± 2.15 | 68.2 ± 1.83 | 68.2 ± 4.66 | 66.4 ± 7.22 |
| | Median | 70 | 68 | 71 | 66 |
| | Min, Max | 66, 71 | 65, 70 | 60, 72 | 56, 78 |

TABLE 24
depicts the Day −1 plasma concentrations (pg/ml) of subjects at different
timepoints during the infusion of dexmedetomidine hydrochloride and normal saline

| | Plasma concentrations (Picograms/ml) | | | | | | | | | | | | | |
|---|---|---|---|---|---|---|---|---|---|---|---|---|---|---|
| | | | | | | | | | | | | | | 30 min |
| | 00:00 | 00:15 | 00:30 | 00:45 | 01:00 | 01:15 | 01:30 | 01:45 | 02:00 | 02:15 | 02:30 | 02:45 | 03:00 | Post |
| Patient No. | Hr | Hr | Hr | Hr | Hr | Hr | Hr | Hr | Hr | Hr | Hr | Hr | Hr | Infusion |
| OP-1 | BLQ | 11.33 | 19.81 | 0 | 0 | 0 | 0 | 0 | 0 | 0 | 0 | 0 | 0 | 5.37 |
| (Treatment) | | | | | | | | | | | | | | |
| OP-2 | BLQ | BLQ | 26.16 | 27.96 | 56.99 | 0 | 0 | 0 | 0 | 0 | 0 | 0 | 0 | 32.44 |
| (Treatment) | | | | | | | | | | | | | | |
| OP-3 | BLQ | 138.2 | 117.44 | 168.47 | 0 | 0 | 0 | 0 | 0 | 0 | 0 | 0 | 0 | 104.11 |
| (Treatment) | | | | | | | | | | | | | | |
| OP-4 | 0 | 0 | 0 | 0 | 0 | 0 | 0 | 0 | 0 | 0 | 0 | 0 | 0 | 0 |
| (Placebo) | | | | | | | | | | | | | | |
| OP-5 | 0 | 0 | 0 | 0 | 0 | 0 | 0 | 0 | 0 | 0 | 0 | 0 | 0 | 0 |
| (Placebo) | | | | | | | | | | | | | | |
| OP-6 | BLQ | 112.58 | 88.23 | 0 | 0 | 0 | 0 | 0 | 0 | 0 | 0 | 0 | 0 | 70.81 |
| (Treatment) | | | | | | | | | | | | | | |
| OP-7 | BLQ | 72.41 | 0 | 0 | 0 | 0 | 0 | 0 | 0 | 0 | 0 | 0 | 0 | 36.58 |
| (Treatment) | | | | | | | | | | | | | | |
| OP-8 | 0 | 0 | 0 | 0 | 0 | 0 | 0 | 0 | 0 | 0 | 0 | 0 | 0 | 0 |
| (Placebo) | | | | | | | | | | | | | | |
| OP-9 | BLQ | BLQ | 25.93 | 66.65 | 61.06 | 75.3 | 0 | 0 | 0 | 0 | 0 | 0 | 0 | 83.87 |
| (Treatment) | | | | | | | | | | | | | | |
| OP-10 | BLQ | 8.39 | 0 | 0 | 0 | 0 | 0 | 0 | 0 | 0 | 0 | 0 | 0 | 13.13 |
| (Treatment) | | | | | | | | | | | | | | |
| OP-11 | BLQ | BLQ | 25.68 | 100.6 | 161.84 | BLQ | 0 | 0 | 0 | 0 | 0 | 0 | 0 | 66.09 |
| (Treatment) | | | | | | | | | | | | | | |
| OP-12 | BLQ | BLQ | BLQ | 8.97 | 45.8 | 110.49 | 111.99 | 0 | 0 | 0 | 0 | 0 | 0 | 68.63 |
| (Treatment) | | | | | | | | | | | | | | |
| OP-13 | 0 | 0 | 0 | 0 | 0 | 0 | 0 | 0 | 0 | 0 | 0 | 0 | 0 | 0 |
| (Placebo) | | | | | | | | | | | | | | |
| OP-14 | 0 | 0 | 0 | 0 | 0 | 0 | 0 | 0 | 0 | 0 | 0 | 0 | 0 | 0 |
| (Placebo) | | | | | | | | | | | | | | |
| OP-15 | BLQ | BLQ | 0 | 0 | 0 | 0 | 0 | 0 | 0 | 0 | 0 | 0 | 0 | B |
| (Treatment) | | | | | | | | | | | | | | |
| OP-17 | BLQ | 10.89 | 53.8 | 46.72 | 36.68 | 0 | 0 | 0 | 0 | 0 | 0 | 0 | 0 | 44.17 |
| (Treatment) | | | | | | | | | | | | | | |

BLQ—Below limit of quantification

TABLE 25
depicts the Day −2 treatment was given to five patients (Patient nos. 1, 2, 3, 9 and 17) depending on the
infusion rate. On Day −1 maximum dexmedetomidine concentrations ranged from about 11.33 pg/ml to about 168.47
pg/ml and on Day 2, maximum dexmedetomidine concentrations ranged from about 23.28 pg/ml to about 403.80 pg/ml

| | Plasma concentrations (picograms/ml) | | | | | | | | | | | | | |
|---|---|---|---|---|---|---|---|---|---|---|---|---|---|---|
| | | | | | | | | | | | | | | 30 min |
| | 00:00 | 00:15 | 00:30 | 00:45 | 01:00 | 01:15 | 01:30 | 01:45 | 02:00 | 02:15 | 02:30 | 02:45 | 03:00 | Post |
| Patient No. | Hr | Hr | Hr | Hr | Hr | Hr | Hr | Hr | Hr | Hr | Hr | Hr | Hr | Infusion |
| OP-1 | BLQ | BLQ | 23.28 | 0 | 0 | 0 | 0 | 0 | 0 | 0 | 0 | 0 | 0 | 9.67 |
| (Treatment) | | | | | | | | | | | | | | |
| OP-2 | BLQ | 22.53 | 25.23 | 42.52 | 54.81 | 0 | 0 | 0 | 0 | 0 | 0 | 0 | 0 | 847.27 |
| (Treatment) | | | | | | | | | | | | | | |
| OP-3 | BLQ | 7.59 | 41.83 | 103.79 | 0 | 0 | 0 | 0 | 0 | 0 | 0 | 0 | 0 | 62.63 |
| (Treatment) | | | | | | | | | | | | | | |
| OP-9 | BLQ | 75.8 | 67.33 | 110.58 | 97.92 | 140.26 | 177.36 | 287.5 | 403.8 | 0 | 0 | 0 | 0 | BLQ |
| (Treatment) | | | | | | | | | | | | | | |
| OP-17(Treatment) | 8.21 | 27.22 | 33.95 | 70.82 | 119.71 | 0 | 0 | 0 | 0 | 0 | 0 | 0 | 0 | 106.39 |

BLQ—Below limit of quantification

Mean infusion rate on Day 1 is 0.29 mcg/kg/hr with the maximum rate ranging from 0.2 mcg/kg/hr to 0.4 mcg/kg/hr.

Mean infusion rate on Day 2 is about 0.44 mcg/kg/hr with the maximum rate ranging from about 0.2 mcg/kg/hr to about 0.7 mcg/kg/hr.

On Day 1, time to reach maximum plasma concentration ranged from about 15 minutes to about 105 minutes.

On Day 2, time to reach maximum plasma concentration ranged from about 30 minutes to about 120 minutes.

TABLE 26
Summary of data for the effect of continuous infusion of dexmedetomidine hydrochloride
in human volunteers, schizophrenia, SDAT and opioid withdrawal patients

| | Healthy | | Healthy | | | | | | Opioid | |
|---|---|---|---|---|---|---|---|---|---|---|
| | Volunteers | | Volunteers | | Alzheimer's | | Schizophrenia | | withdrawal | |
| Parameter | Cohort 1 | | Cohort 2 | | Study | | Study | | Study | |
| Range of | 0.3-0.5 | mcg/kg/hr | 0.3-0.4 | mcg/kg/hr | 0.2-0.5 | mcg/kg/hr | 0.2-0.4 | mcg/kg/hr | 0.2-0.7 | mcg/kg/hr |
| efficacious dose of | | | | | | | | | | |
| IV | | | | | | | | | | |
| dexmedetomidine | | | | | | | | | | |
| hydrochloride | | | | | | | | | | |
| Total dose | 21-79 | mcg | 12-33 | mcg | 7-34 | mcg | 5-32 | mcg | 3.2-83.6 | mcg |
| Efficacy | 6/6 RASS-1 | | 5/6 RASS-1 | | 7/10 RASS-1 | | 9/10 RASS-1 | | 11/11 COWS | |
| (Primary/secondary | | | | | | | and | | greater than or | |
| end points) | | | | | | | 9/10 PEC score | | equal to 50% | |
| | | | | | | | below 7 | | reduction | |
| Plasma | 114-632 | pg/ml | 119-363 | pg/ml | 102-334 | pg/ml | 22-406 | pg/ml | 11-406 | pg/ml |
| concentration | | | | | | | | | | |
| achieved at | | | | | | | | | | |
| efficacious doses | | | | | | | | | | |

| | | | | | |
|---|---|---|---|---|---|
| Hemodynamic | Systolic BP | All | All | All | In one patient |
| effects | fall >20 mmHg | hemodynamic | hemodynamic | hemodynamic | (#007) HR |
| | in three patients | parameters | parameters | parameters | reduced |
| | | remained within | remained within | remained within | |
| | | normal limits | normal limits | normal limits | |

1. A method for treating agitation or signs of agitation in a human subject who is alert prior to treatment, comprising administering dexmedetomidine hydrochloride intravenously to the subject at a cumulative dose of about 0.1 mcg/kg to about 1 mcg/kg to achieve the desired anti-agitation effect.

2. A method for treating agitation or signs of agitation in a human subject who is alert prior to treatment, comprising administering dexmedetomidine hydrochloride intravenously to the subject to a total dose in a range from about 2 mcg to about 100 mcg to achieve the desired anti-agitation effect.

3. The method according to claim 1 or claim 2 for the treatment of acute agitation.

4. The method according to any preceding claim, wherein the agitation or signs of agitation are associated with senile dementia of the Alzheimer's type (SDAT), Alzheimer's disease, schizophrenia, opioid withdrawal disorder, bipolar disorder, or hyperactive delirium.

5. The method according to claim 4, wherein the agitation or signs of agitation is associated with senile dementia of the Alzheimer type (SDAT).

6. The method according to claim 4, wherein the agitation or signs of agitation is associated with schizophrenia.

7. The method according to claim 4, wherein the agitation or signs of agitation is associated with opioid withdrawal.

8. The method according to any preceding claim, wherein the desired anti-agitation effect is achieved without also producing clinically meaningful effects on blood pressure and/or heart rate.

9. The method according to any preceding claim, wherein dexmedetomidine hydrochloride is administered intravenously as one or more discrete injections.

10. The method according to any one of claims 1 to 8, wherein dexmedetomidine hydrochloride is administered intravenously as a continuous infusion.

11. The method according to claim 10, wherein the infusion is given at a rate of about 0.1 to about 0.6 mcg/kg/hr.

12. The method according to any preceding claim, wherein the subject is between 55 to 75 years old.

13. The method according to any preceding claim, wherein agitation is reduced to a value of 7 or below 7 according to the PEC scale.

14. The method according to any preceding claim, wherein the reduction in agitation is associated with the attainment of a mild sedative effect.

15. The method according to claim 14, wherein the mild sedative effect corresponds to a RASS score of −1.

16. The method according to claim 14, wherein the mild sedative effect persists for up to about 120 minutes.

17. A method of treating agitation or signs of agitation in a human subject suffering from senile dementia of the Alzheimer's type (SDAT) who is alert prior to treatment, comprising administering dexmedetomidine hydrochloride intravenously to the subject at a cumulative dose of about 0.15 mcg/kg to about 0.75 mcg/kg to achieve the desired anti-agitation effect, which effect persists for up to about 30 minutes after discontinuation of administration of dexmedetomidine hydrochloride, and wherein the reduction in agitation is associated with the attainment of a mild sedative effect.

18. A method of treating agitation or signs of agitation in a human subject suffering from senile dementia of the Alzheimer's type (SDAT) who is alert prior to treatment, comprising administering dexmedetomidine hydrochloride intravenously to the subject at a total dose of about 5 mcg to about 40 mcg to achieve the desired anti-agitation effect, which effect persists for up to about 30 minutes after discontinuation of administration of dexmedetomidine hydrochloride, and wherein the reduction in agitation is associated with the attainment of a mild sedative effect.

19. The method according to claim 17 or claim 18, wherein the mild sedative effect corresponds to a RASS score of −1.

20. The method according to any one of claims 17 to 19, wherein the subject is between 55 to 75 years old.

21. The method according to any one of claims 17 to 20, wherein the desired anti-agitation effect is achieved without also producing clinically meaningful effects on blood pressure and/or heart rate.

22. The method according to any one of claims 17 to 21, wherein dexmedetomidine hydrochloride is administered intravenously as one or more discrete injections.

23. A method of treating agitation or signs of agitation in a human subject suffering from schizophrenia who is alert prior to treatment, comprising administering dexmedetomidine hydrochloride intravenously to the subject at a total dose of about 5 mcg to about 40 mcg to achieve the desired anti-agitation effect, wherein agitation is reduced to a value of 7 or below 7 according to the PEC scale, and wherein the reduction in agitation is associated with the attainment of a mild sedative effect.

24. The method according to claim 23, wherein the mild sedative effect corresponds to a RASS score of −1.

25. The method according to claim 23 or claim 24, wherein the desired anti-agitation effect is achieved without also producing clinically meaningful effects on blood pressure and/or heart rate.

26. The method according to any one of claims 23 to 25, wherein dexmedetomidine hydrochloride is administered intravenously as one or more discrete injections.

27. A method of treating agitation or signs of agitation in a human subject suffering from opioid withdrawal disorder who is alert prior to treatment, comprising administering dexmedetomidine hydrochloride intravenously to the subject at a total dose of about 3 mcg to about 90 mcg to achieve at least a 50% reduction in COW score.

28. The method according to claim 27, wherein the desired anti-agitation effect is achieved without also producing clinically meaningful effects on blood pressure and/or heart rate.

29. The method according to claims 27 to 28, wherein dexmedetomidine hydrochloride is administered intravenously as one or more discrete injections.

30. The method according to claims 27 to 28, wherein dexmedetomidine hydrochloride is administered intravenously as a continuous infusion.

31. The method according to claim 30, wherein the dose is about 0.3 mcg/kg/hr to 0.4 mcg/kg/hr.

32. A method of treating agitation or signs of agitation in a human subject suffering from senile dementia of the Alzheimer's type (SDAT) who is alert prior to treatment, comprising administering to the subject dexmedetomidine hydrochloride intravenously by continuous infusion at an infusion rate up to about 0.5 mcg/kg/hr to achieve a maximum plasma concentration (Cₘₐₓ) of up to about 400 pg/ml, together with the attainment of a mild sedative effect.

33. A method of treating agitation or signs of agitation in a human subject suffering from schizophrenia who is alert prior to treatment, comprising administering to the subject dexmedetomidine hydrochloride intravenously by continuous infusion at an infusion rate up to about 0.5 mcg/kg/hr to achieve a maximum plasma concentration (Cₘₐₓ) of up to about 400 pg/ml, wherein agitation is reduced to a value of 7 or below 7 according to the PEC scale, and wherein the reduction in agitation is associated with the attainment of a mild sedative effect.

34. A method of treating agitation or signs of agitation in a human subject suffering from opioid withdrawal disorder who is alert prior to treatment, comprising administering to the subject dexmedetomidine hydrochloride intravenously by continuous infusion at an infusion rate up to about 0.7 mcg/kg/hr to achieve a maximum plasma concentration (Cₘₐₓ) of up to about 410 pg/ml, and at least a 50% reduction in COW score.
